(19) United States
(12) Patent Application Publication
(10) Pub. No.: US 2026/0083376 A1
(43) Pub. Date: Mar. 26, 2026

(54) BRAIN-COMPUTER INTERFACE SYSTEM AND METHOD

(71) Applicant: Science Corporation, Alameda, CA (US)

(72) Inventors: Marat Rostov, Alameda, CA (US); Nate Slager, Alameda, CA (US); Sage Walker, Alameda, CA (US); Max Hodak, Alameda, CA (US); Russell Sharpe, Alameda, CA (US); Emma Zhou, Alameda, CA (US); Antonia Elsen, Alameda, CA (US)

(73) Assignee: Science Corporation, Alameda, CA (US)

(21) Appl. No.: 19/336,033

(22) Filed: Sep. 22, 2025

Related U.S. Application Data
(60) Provisional application No. 63/696,980, filed on Sep. 20, 2024.
(60) Provisional application No. 63/764,784, filed on Feb. 28, 2025.
(60) Provisional application No. 63/799,070, filed on May 2, 2025.

(51) Int. Cl. A61B 5/293; A61B 5/00; A61B 5/263

(52) U.S. Cl. CPC A61B 5/293; A61B 5/0006; A61B 5/263; A61B 5/4836

(57) ABSTRACT

Variants of the system can include: a probe and an interface device. Variants of the method can include: configuring a signal pipeline and executing the signal pipeline. In variants, the system and/or method can function to record neural signals from a variety of brain-computer interface (BCI) probe devices. In a specific example, the system and/or method can enable high bandwidth neural recording and processing for BCI experiments.

### CROSS-REFERENCE TO RELATED APPLICATIONS

This application claims the benefit of U.S. Provisional Application No. 63/799,070 filed 2 May 2025, U.S. Provisional Application No. 63/764,784 filed 28 Feb. 2025, and U.S. Provisional Application No. 63/696,980 filed 20 Sep. 2024, each of which is incorporated in its entirety by this reference.

### TECHNICAL FIELD

This invention relates generally to the brain-computer interface field, and more specifically to a new and useful system and method in the brain-computer interface field.

### BRIEF DESCRIPTION OF THE SEVERAL VIEWS OF THE DRAWINGS

FIG. 1A is a schematic representation of a first variant of the system.

FIG. 1B is a schematic representation of a second variant of the system.

FIG. 2 is a schematic representation of a variant of the method.

FIG. 3A depicts a swim lane representation of a variant method.

FIG. 3B depicts a swim lane representation of another variant method, including an adapter.

FIGS. 4A-4C depict illustrative examples of signal pipeline visualizations.

FIG. 5A depicts a top-down view of a specific example of the interface device.

FIG. 5B depicts a side view of a specific example of the interface device.

FIG. 6 depicts an example of the interface device.

FIG. 7 depicts another example of the interface device.

FIGS. 8A and 8B depict specific examples of a display on the interface device.

FIG. 9 depicts a specific example of the system, including an adapter.

FIG. 10 depicts another specific example of the system, including an adapter.

FIG. 11 depicts a specific example of the interface device.

FIG. 12 depicts a specific example of the method, including a user-defined node.

FIG. 13A depicts a specific example of circuitry connected to the neural probe (e.g., local to the neural probe and/or local to an adapter connected to the neural probe).

FIG. 13B depicts a specific example of switches (e.g., programmable switches) connected to the neural probe (e.g., local to the neural probe and/or local to an adapter connected to the neural probe).

FIGS. 14A-14C depict illustrative examples of a neural probe.

FIG. 15A-15U depict illustrative examples of a user interface.

### DETAILED DESCRIPTION OF THE INVENTION

The following description of the embodiments of the invention is not intended to limit the invention to these embodiments, but rather to enable any person skilled in the art to make and use this invention.

### 1. Overview

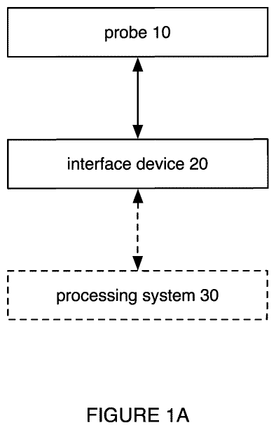

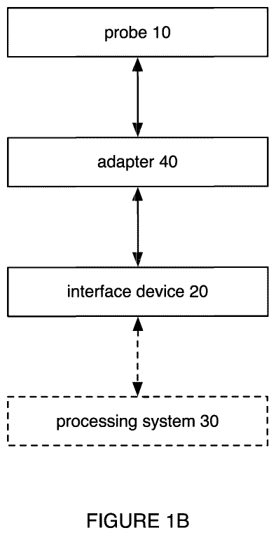

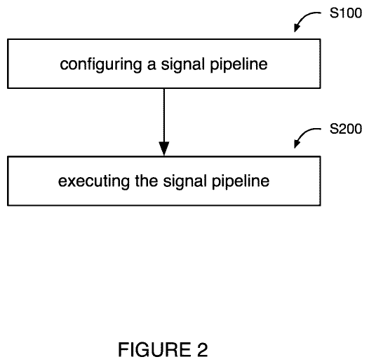

As shown in FIG. 1A and FIG. 1B, the system can include: a probe 10 and an interface device 20. However, the system can additionally or alternatively include any other suitable elements. As shown in FIG. 2, the method can include: configuring a signal pipeline S100 and executing the signal pipeline S200. However, the method can additionally or alternatively include any other suitable steps.

In variants, the system and/or method can function to record neural signals from a variety of brain-computer interface (BCI) probe devices. In a specific example, the system and/or method can enable high bandwidth neural recording and processing for BCI experiments.

### 2. Examples

In an example, the system can include: a neural probe and an interface device (e.g., a headstage device). In a first specific example, the neural probe connects directly to the interface device. In a second specific example, the neural probe connects to an adapter connected to the interface device. The interface device can optionally configure the neural probe and/or the adapter based on user-provided neural probe specifications (e.g., an electrode arrangement). For example, the neural probe and/or the adapter can include a set of programmable switches connecting a set of electrodes to a set of recording channels; the interface device can control the set of programmable switches based on the neural probe specifications to connect two electrodes to each recording channel of the set of recording channels. In a specific example, each recording channel can be connected to a first electrode specified in the electrode arrangement as a recording electrode, and a second electrode specified in the electrode arrangement as a reference electrode. The reference electrode can be the same across all recording channels (e.g., a global reference mode), or can be different (e.g., a differential recording mode, where either electrode can record neural signals).

In a specific example, a user can configure a signal pipeline defined by an API, wherein the signal pipeline includes a sequence of nodes for data communication (e.g., recording neural signals, transmitting signals), signal processing (e.g., filtering neural signals, spike detection), signal generation (e.g., determining electrical and/or optical stimulus signals), and/or any other signal pipeline elements. The interface device can optionally execute the signal pipeline. In a first specific example, the signal pipeline can include a recording node, wherein the interface device controls the neural probe to collect and transmit the neural signals to the interface device (e.g., using a defined data transport protocol). In a second specific example, the signal pipeline can include a processing node, wherein the interface device processes the recorded neural signals (e.g., filtering, detecting spikes, etc.). In a third specific example, the signal pipeline can include a stimulus generation node, wherein the interface device determines stimulus signals (e.g., based on processed or unprocessed neural signals). In a fourth specific example, the signal pipeline can include a stimulation node, wherein the interface device controls the neural probe to apply stimulus signals (e.g., electrical signals and/or optical signals). In a fifth specific example, the signal pipeline can include a data transfer node, wherein the interface device transmits signals (e.g., process or unprocessed neural signals, stimulation signals, etc.) to an external device (e.g., the neural probe, a remote processing system, etc.)

Configuring the signal pipeline can optionally include defining a reference mode for the neural probe. In a first specific example, in a global reference mode, a single electrode of the neural probe can be assigned a reference electrode identifier and remaining electrodes of the neural probe can be assigned a recording electrode identifier. In a second specific example, in a differential reference mode, the electrodes can be grouped into pairs, wherein one electrode of each pair is assigned a reference electrode identifier and the other electrode is assigned a recording electrode identifier. In an illustrative example, for a pair of electrodes, Electrode A and Electrode B, a user can assign Electrode A a recording electrode identifier and assign Electrode B a reference electrode identifier, wherein the sign of a differential signal recorded from Electrode A and Electrode B (e.g., the signal from Electrode A referenced to Electrode B) can indicate the origin of the neural signal. For example, when a neural signal is received at Electrode A, the differential signal is positive, and when a neural signal is received at Electrode B, the differential signal is negative. The recording node can optionally be configured based on the assigned reference electrode identifiers and recording electrode identifiers. However, the system and/or method can be otherwise performed.

### 3. Technical Advantages

Variants of the technology can confer one or more advantages over conventional technologies.

In an example, variants of the technology can provide a standard interface for interacting with a range of different neural probes and/or probe configurations. In a first example, the standard interface (e.g., an interface device and/or an API defining interactions between the interface device and one or more probes) can interact with both unidirectional probes (e.g., functioning to read from a set of neurons using electrodes) and bidirectional probes (e.g., functioning to read from and write to a set of neurons using electrodes and/or LEDs). In a second example, the standard interface can interact with probes configured in different reference modes. In a specific example, the standard interface can interact with probes configured in a global reference mode (e.g., the probe includes a single reference electrode) and/or probes configured in a differential reference mode (e.g., electrodes on the probe are grouped into pairs, wherein either electrode in the pair of electrode can act as a reference for the other electrode when the other electrode receives a neural pulse).

However, further advantages can be provided by the system and method disclosed herein.

### 3. System

As shown in FIG. 1A and FIG. 1B, the system can include: a probe 10 and an interface device 20. The system can optionally include a processing system 30 (e.g., as part of the interface device 20 and/or remote from the interface device 20), an adapter 40, and/or any other suitable elements.

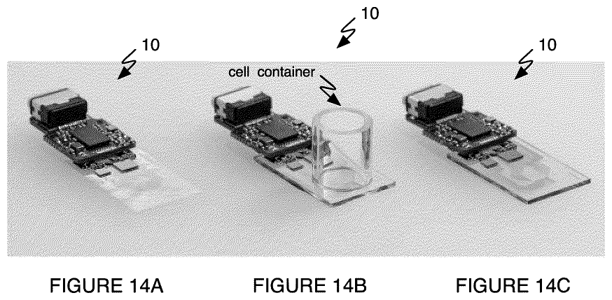

The probe 10 (e.g., neural probe) functions to receive signals from and/or send signals to a set of cells. Examples are shown in FIG. 14A, FIG. 14B, and FIG. 14C. The probe 10 can additionally or alternatively function to apply electrical signals to electrodes to determine the electrical interface quality. In a first example, the probe can be a unidirectional probe functioning to read from a set of neurons (e.g., recording electrical signals). In a second example, the probe can be a bidirectional probe functioning to read from and write to a set of neurons (e.g., recording electrical signals and transmitting light signals and/or electrical signals).

The probe 10 preferably directly or indirectly interfaces with neurons, but can additionally or alternatively interface with other cells (e.g., retinal cells, any other somatic cells, reproductive cells, stem cells, plant cells, etc.) and/or any other signal source. In a first specific example, the probe 10 directly interfaces with the set of neurons (e.g., the probe 10 can be implanted in the brain or on the surface of the brain; neurons can be cultured in a well coupled to the probe 10; etc.). In a second specific example, the probe 10 indirectly interfaces with the set of neurons (e.g., the probe 10 can be mounted to the scalp). The neurons can be in vivo (e.g., within a brain), ex vivo, in vitro, and/or otherwise configured. The neurons can be derived from a human, any other mammal (e.g., chimpanzee, rat, rabbit, mouse, etc.), and/or any other organism. In a first specific example, when the neurons are in vivo, all or a portion of the probe 10 (e.g., the head of the probe 10) can be implanted within the brain of an organism or on the surface of the brain of an organism. In a second specific example, the probe 10 (e.g., the head of the probe 10) can include a cell container (e.g., arranged above the set of electrodes), wherein the neurons are cultured within the cell container.

The neurons can optionally be optogenetically modified. In an example, neurons can be genetically modified by transfecting the cells with a light-sensitive protein (e.g., using a virus with a plasmid and capsid) that acts as an optogenetic actuator (e.g., optogenetic effector). In a specific example, optogenetic actuators can produce a biochemical signal (e.g., an action potential) in response to receiving light at a specific wavelength. In a first specific example, the probe 10 can interface with a set of optogenetically modified neurons, wherein the probe 10 can apply a light signal (e.g., wherein the light signal is received by the set of optogenetically modified neurons). In a second specific example, a supplementary device can interface with a set of optogenetically modified neurons, wherein the supplementary device can apply a light signal (e.g., wherein the light signal is received by the set of optogenetically modified neurons). The capsid can be an adeno-associated virus capsid (e.g., AAV2.7M8) and/or any other suitable capsid. The plasmid can be an opsin, a fluorescent biosensor protein, and/or any other suitable plasmid. Opsin examples include: CheRiff, ChroMD, ChroME, ChroME2S, ChRmine (e.g., ChRmine-mScarlet), ChrimsonR (e.g., including red-shifted variants), ReachR, WiChr, a variant of WiChr, and/or any other opsin. Fluorescent biosensor protein examples include: GCaMP8s, GCaMP8m, jRGecola, YCaMP, iGECI, and/or any other suitable protein. The opsin can be activated by blue light, green light, red light, and/or any other wavelength.

The probe 10 preferably includes a set of electrodes (e.g., an array of electrodes) that can directly or indirectly interface with the set of neurons, but can additionally or alternatively include other neural recording methods (e.g., fMRI, fNIRS, ultrasound, etc.). The set of electrodes can optionally include one or more recording electrodes and one or more reference electrodes. Electrodes in the set of electrodes can optionally function as both a recording and a reference electrode. In a specific example, a differential signal can be recorded from a pair of electrodes, and when a signal is received at either electrode, the other can function as the reference electrode. The number of electrodes in the set of electrodes can be between 1-100 million or any range or value therebetween (e.g., at least 100, at least 500, at least 1 k, at least 10 k, at least 50 k, at least 100 k, at least 395 k, at least 1 million, etc.), but can alternatively be greater than 100 million. The number of neurons in the set of neurons can be between 1-100 million or any range or value therebetween (e.g., at least 100, at least 500, at least 1 k, at least 10 k, at least 50 k, at least 100 k, at least 395 k, at least 1 million, etc.), but can alternatively be greater than 100 million.

The probe 10 can optionally include a set of LEDs that can directly or indirectly interface with the set of neurons. In a specific example, the probe 10 can transmit light signals to one or more optogenetically modified neurons. In a first example, the probe 10 can be a unidirectional probe functioning to read from the set of neurons (e.g., recording electrical signals). In a second example, the probe 10 can be a bidirectional probe functioning to read from and write to the set of neurons (e.g., recording electrical signals and transmitting light signals and/or electrical signals). In a specific example, an LED in the set of LEDs on the probe 10 can transmit a light signal to a neuron of interest in the set of neurons, and an electrode in the set of electrodes on the probe 10 can receive an electrical signal from the neuron of interest. In a specific example, the probe 10 can be controlled to drive the set of LEDs based on the set of neural signals and/or stimulation parameters.

The material of the set of LEDs can include indium gallium nitride, gallium nitride (e.g., biocompatible gallium nitride), indium gallium phosphide, an organic emitter, and/or any other light emission materials. The wavelength of the set of LEDs can be between 300 nm-900 nm or any range or value therebetween (e.g., 510 nm-550 nm, approximately 530 nm, 450 nm-485 nm, 500 nm-570 nm, 625 nm-750 nm, etc.). The probe 10 can optionally include one or more microoptics components, which can function to collimate light, homogenize light, focus light (e.g., onto a smaller number of cells), and/or otherwise modify light emitting from the stimulus module 30. Examples of microoptics components can include: lenses (e.g., microlens, diffractive lens, metalens, etc.), back reflectors, a modified LED shape, waveguides, a spacing gap, a combination thereof, and/or any other optics components.

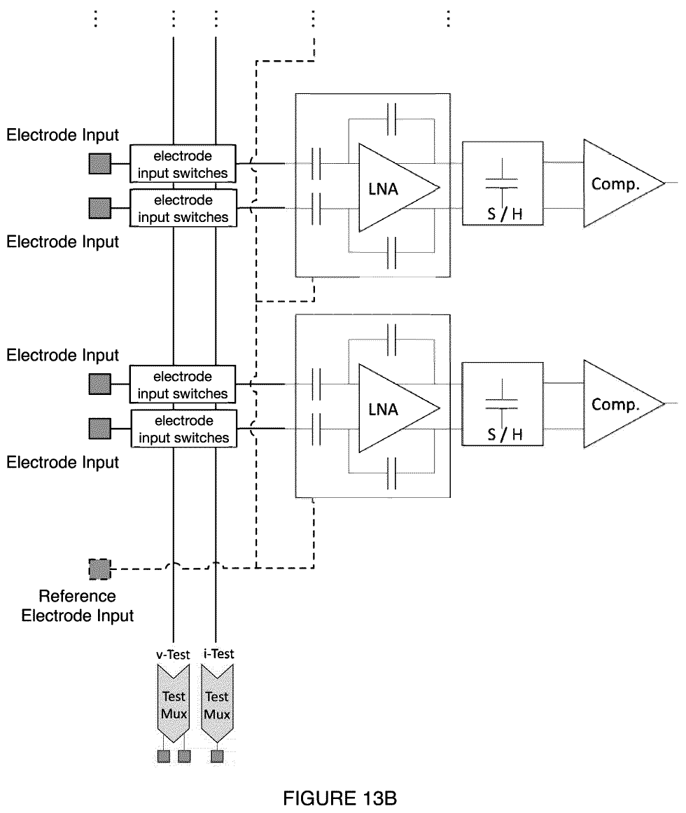

The probe 10 can optionally include and/or interface with an integrated circuit. Examples are shown in FIG. 13A and FIG. 13B. The integrated circuit can optionally include one or more of: a set of electrode inputs, routing resources (e.g., a set of switches), a differential amplifier, an analog memory component, an analog-to-digital converter (ADC) component, optional capacitors, a set of general-purpose input/outputs (GPIOs), and/or any other integrated circuit components. In a specific example, the integrated circuit can include an FPGA. For example, the integrated circuit can include a set of switches (e.g., programmable switches) and a set of recording channels (e.g., differential recording channels). In a specific example, each recording channel can include a differential amplifier (e.g., LNA) configured to record a differential signal. In an example, the set of switches can connect the set of electrodes on the probe 10 to the set of recording channels, where each recording channel is configured to record a differential signal from two electrodes (e.g., a recording electrode and a reference electrode). In a first example, all or a portion of the integrated circuit (e.g., the set of switches and/or the set of recording channels) can be located on the probe 10. In a specific example, the probe 10 can include: a set of electrodes, a set of recording channels, and a set of switches (e.g., programmable switches) connecting the set of electrodes to the set of recording channels. In a second example, all or a portion of the integrated circuit (e.g., the set of switches and/or the set of recording channels) can be located on the adapter 40. In a specific example, the probe 10 can include a set of electrodes, and the adapter 40 can include a set of inputs connected to the set of electrodes of the probe 10; a set of recording channels; and a set of switches (e.g., programmable switches) connecting the set of inputs to the set of recording channels;

In a first variant, the reference electrode is different for each recording channel of the set of recording channels. For example, when the neural probe is configured in a differential reference mode, a different reference electrode is connected to each recording channel of the set of recording channels, where both the recording electrode and the reference electrode for each recording channel are configured to record neural signals. In a specific example, this differential reference mode can operate with the assumption that only one active neural pulse will come from one of the two inputs of the recording channel (one of the two electrodes), while the other idle input can act as a reference. In an illustrative example, when the sign of a neural signal recorded from a recording channel is negative, the neural signal corresponds to the reference electrode (e.g., the neural signal originated at a neuron interfacing with the reference electrode); when the sign of a neural signal recorded from a recording channel is positive, the neural signal corresponds to the recording electrode (e.g., the neural signal originated at a neuron interfacing with the recording electrode). In a second variant, the reference electrode is the same for each recording channel of the set of recording channels. For example, when the neural probe is configured in a global reference mode, a single reference electrode is connected to each recording channel of the set of recording channels.

The probe 10 can optionally include systems as described in U.S. application Ser. No. 19/097,738 filed 1 Apr. 2025 and/or U.S. application Ser. No. 19/097,539 filed 1 Apr. 2025, each of which is incorporated in its entirety by this reference.

However, the probe 10 can be otherwise configured.

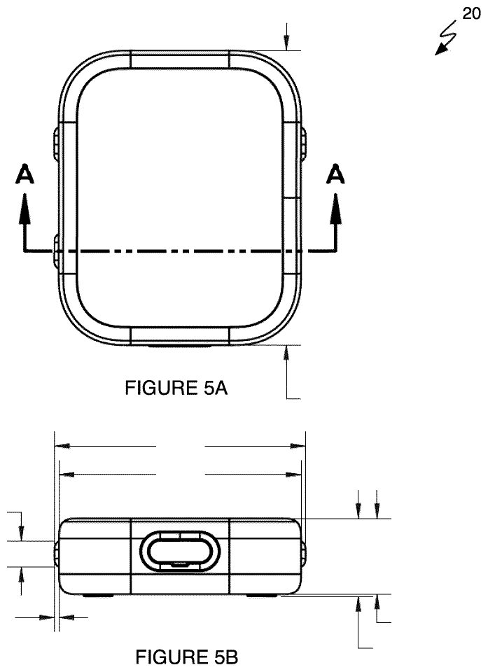

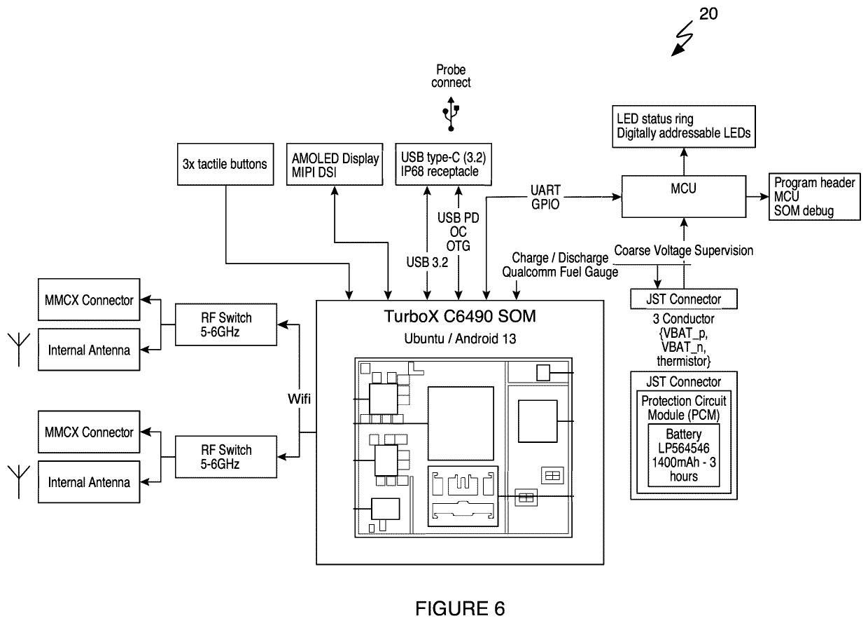

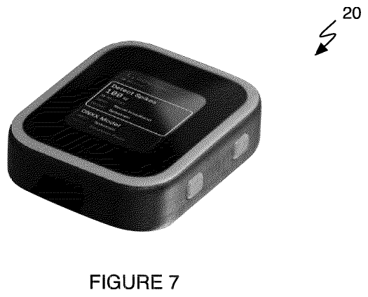

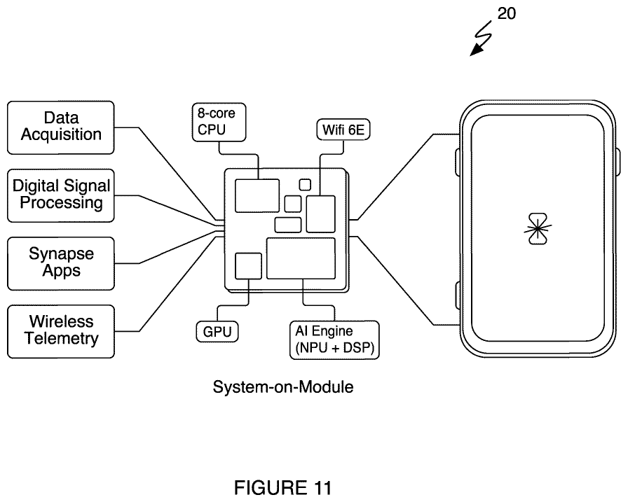

The interface device 20 functions to interface with the probe 10, receiving neural signals from the probe 10 and/or transmitting stimulus signals to the probe 10. Additionally or alternatively, the interface device 20 (e.g., a headstage device) can function to: control the probe 10, configure the probe 10, process neural signals, generate stimulus signals, and/or communicate with an external processing system. Specific examples of the interface device 20 are shown in FIG. 5A, FIG. 5B, FIG. 6, FIG. 7, and FIG. 11. The interface device 20 can be connected to: one or more probes 10, one or more adapters 40, one or more processing systems 30 (e.g., the interface device 20 can be communicatively connected to one or more processing systems 30), one or more supplementary devices, and/or any other system components.

In a first variant, the interface device 20 can interface with a single probe 10. In a second variant, the interface device 20 can interface with multiple probes 10 (e.g., at least two probes, at least 3, at least 5, at least 10, etc.). The interface device 20 can optionally be configured to interface with different probe types (e.g., unidirectional and bidirectional probes).

The interface device 20 can optionally interface with one or more supplementary devices (e.g., interface directly, interface via the adapter 40, etc.). Examples of supplementary devices include: measurement devices (e.g., temperature sensor, current sense resistors, pressure sensor, chemical sensor, humidity sensor, multimeter, etc.), telemetry components, user interfaces, processing systems, stimulation devices (e.g., configured to deliver stimulus signals), LEDs, a user device, joystick, keyboard, mouse, space mouse, and/or any other devices. In a specific example, the supplementary device can be a behavior recorder, configured to record a user action. The supplementary devices can optionally transmit data (e.g., signals) to the interface device 20. Examples of data include: measurements, user inputs, test signals, and/or any other data.

However, the interface device 20 can be otherwise configured.

The system can optionally include a processing system 30, which can function to receive user inputs and/or execute all or a portion of the signal pipeline (e.g., as described below). The processing system 30 can include one or more: CPUs, GPUs, TPUs, custom FPGA/ASICS, microprocessors, servers, cloud computing, and/or any other suitable components. The processing system 30 can be local, remote (e.g., cloud computing server, etc.), distributed, and/or otherwise arranged relative to any other system or module. In a first example, the processing system 30 can include one or more local processing systems, local to the neural probe 10, the interface device 20, and/or the adapter 40. In a second example, the processing system 30 can optionally include one or more remote processing systems. In a third example, the processing system 30 can include one or more local processing systems and one or more remote processing systems. However, the processing system 30 can be otherwise configured.

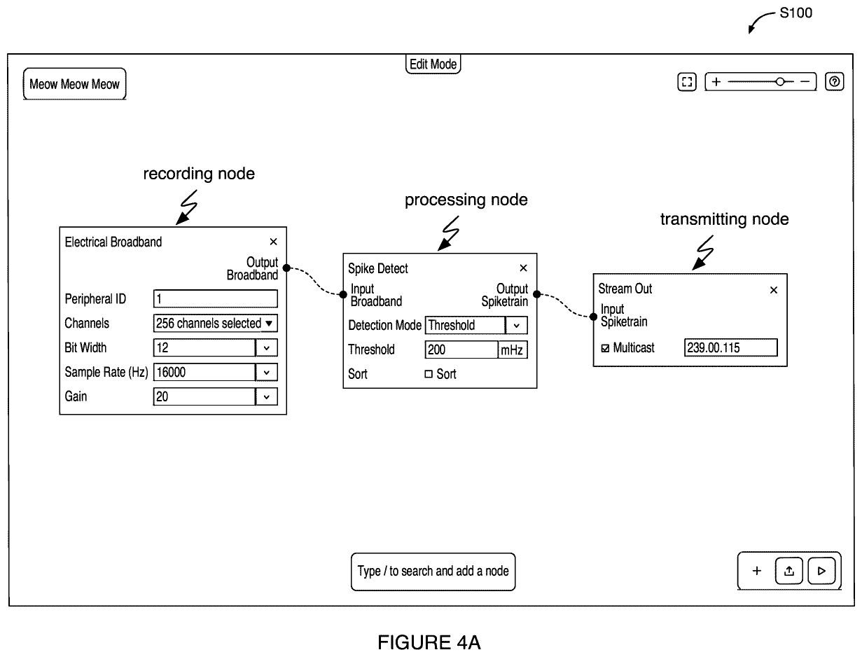

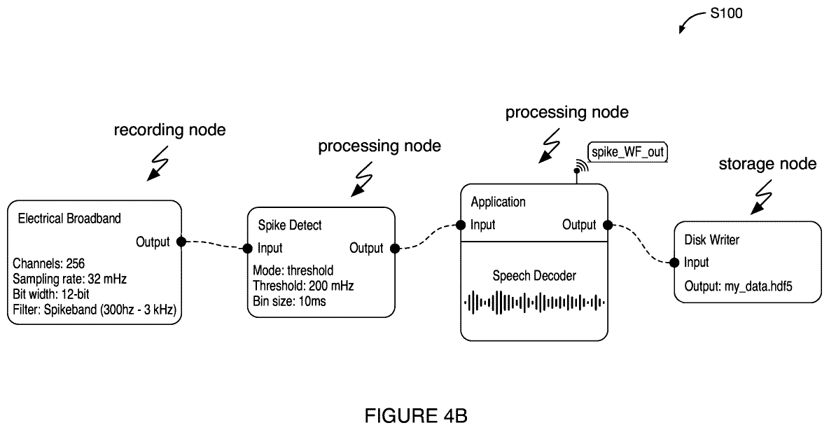

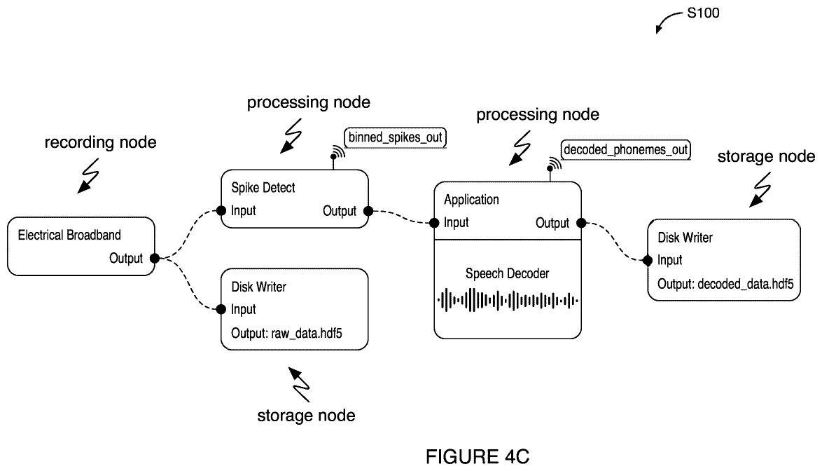

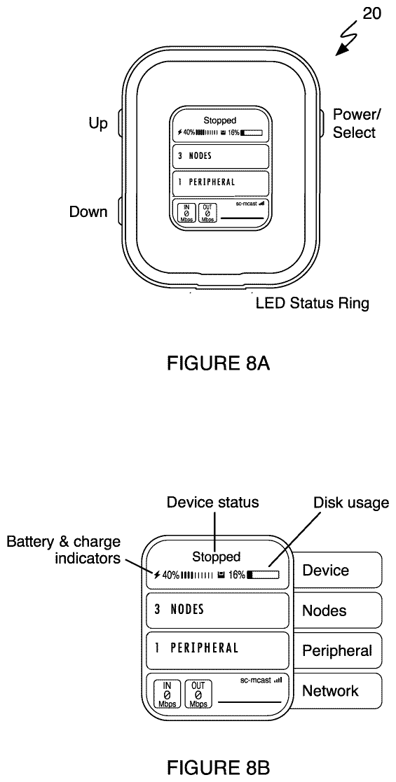

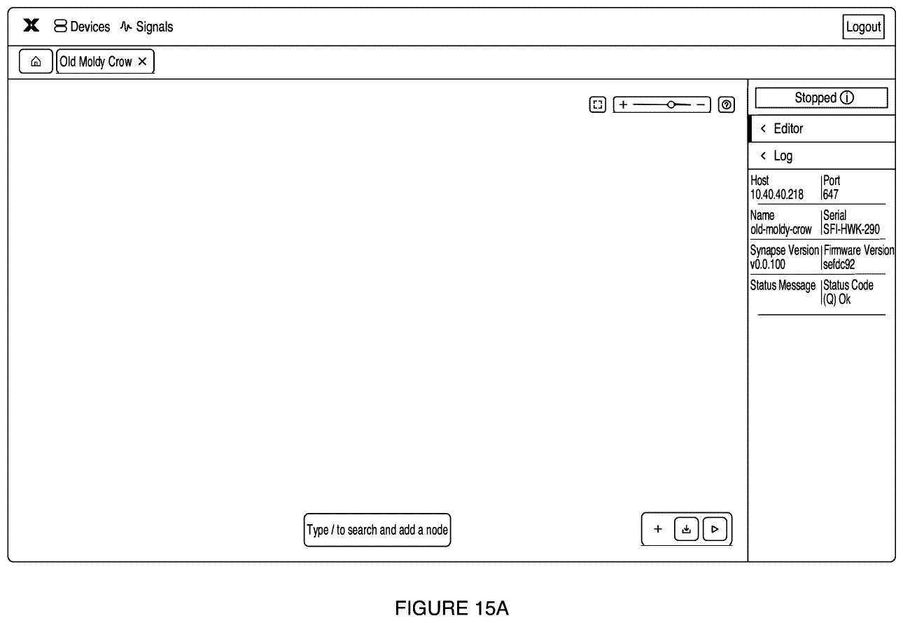

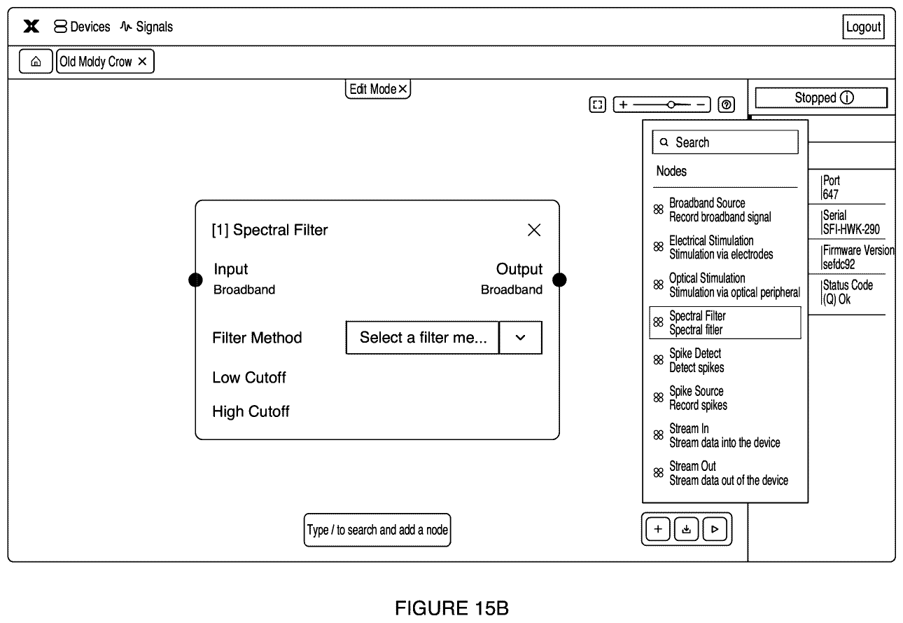

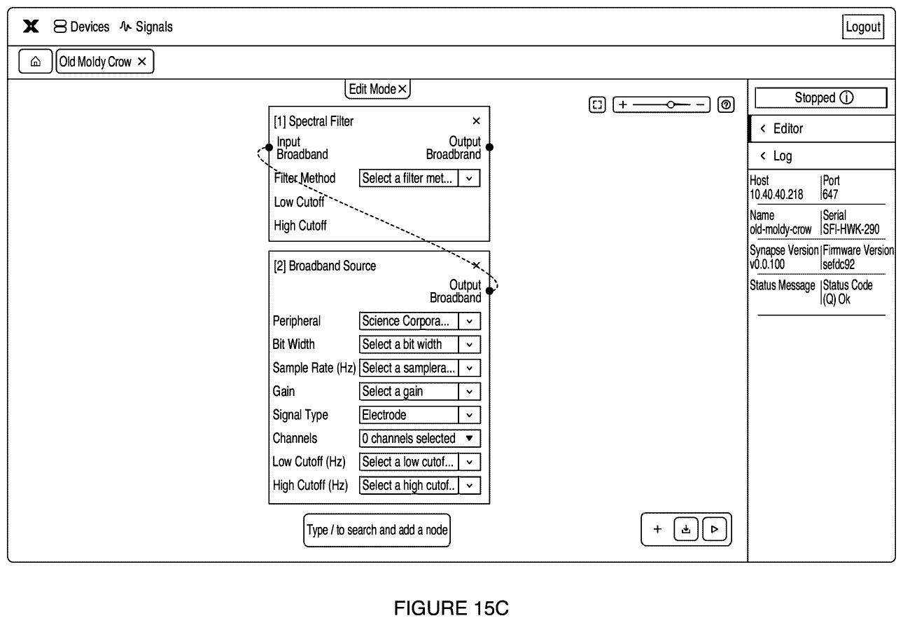

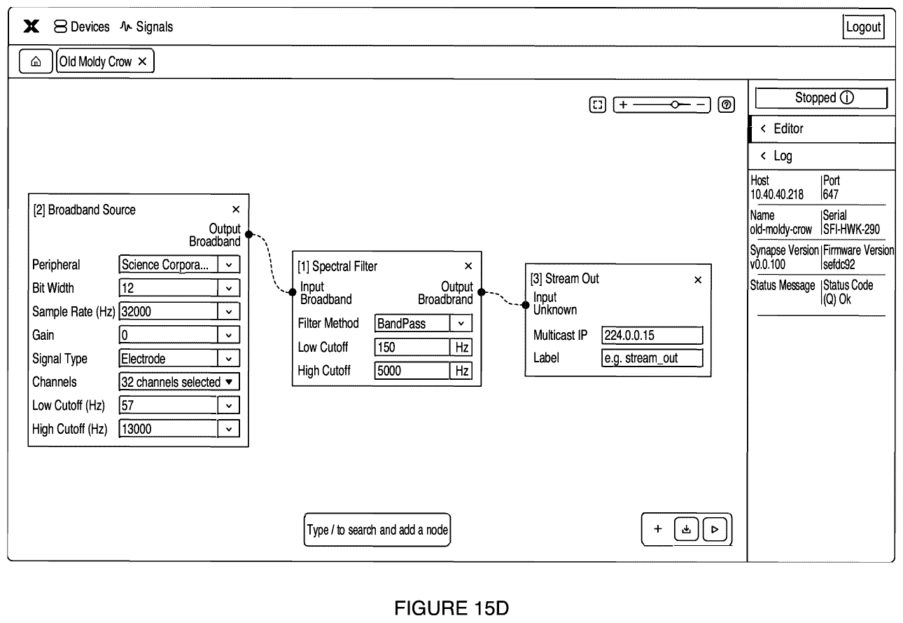

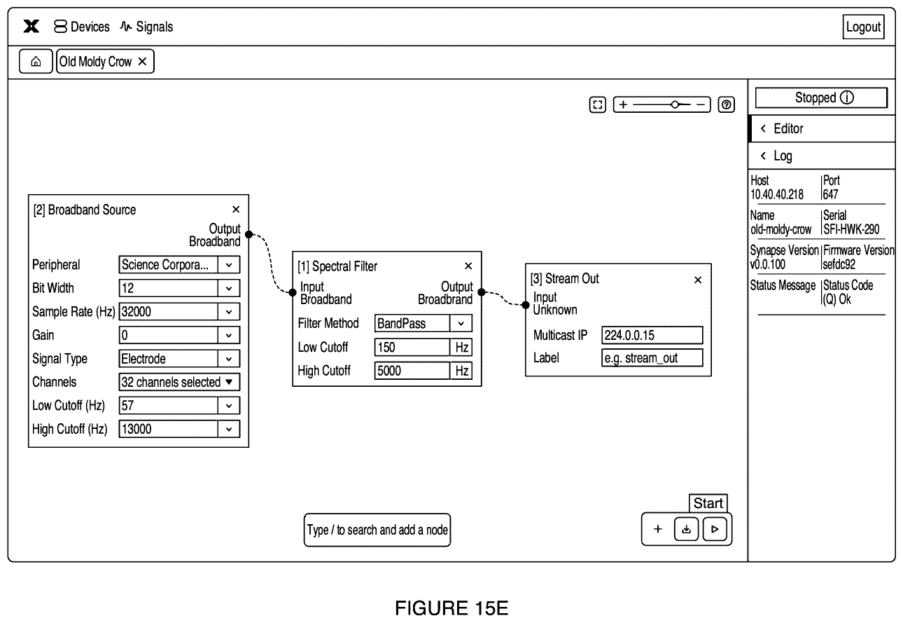

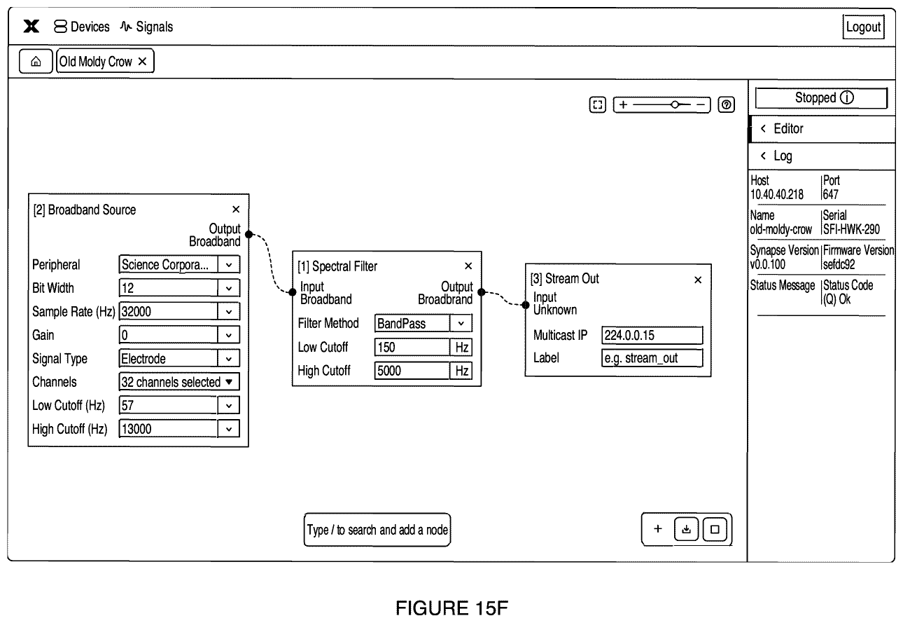

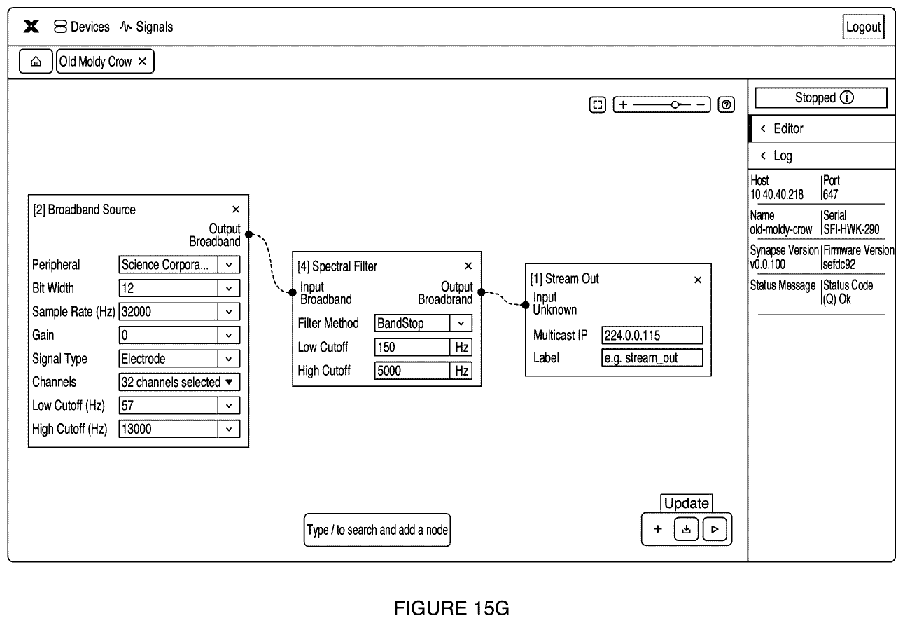

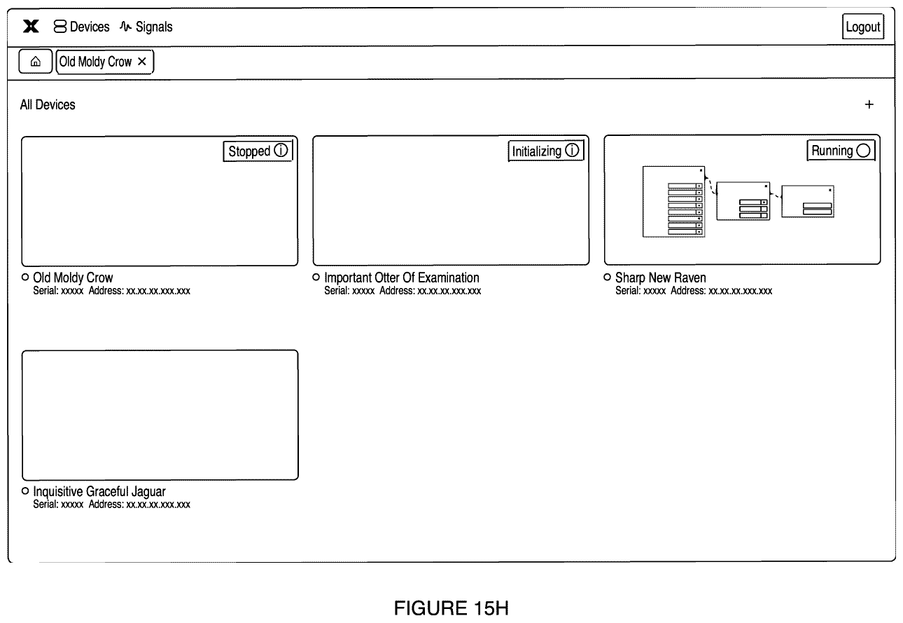

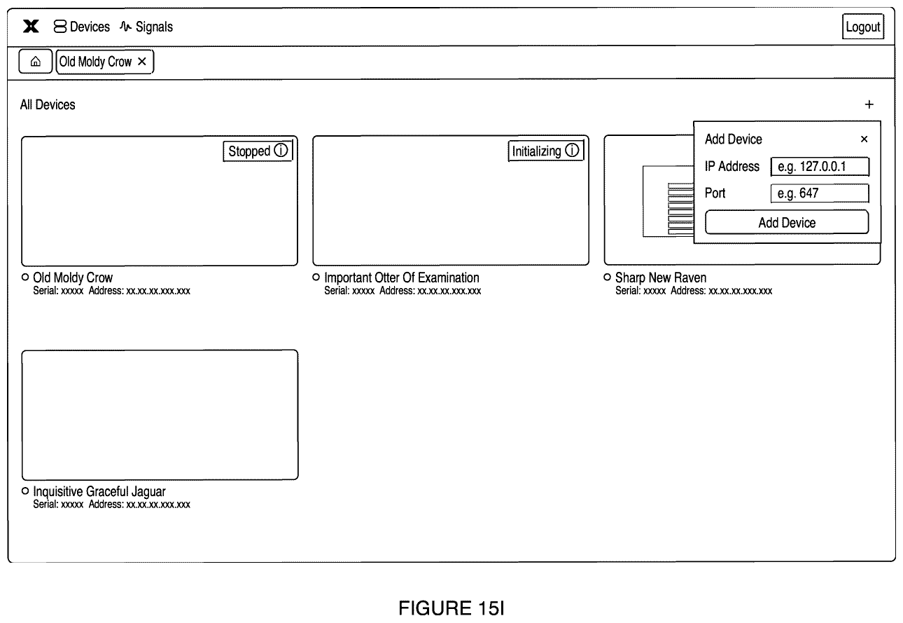

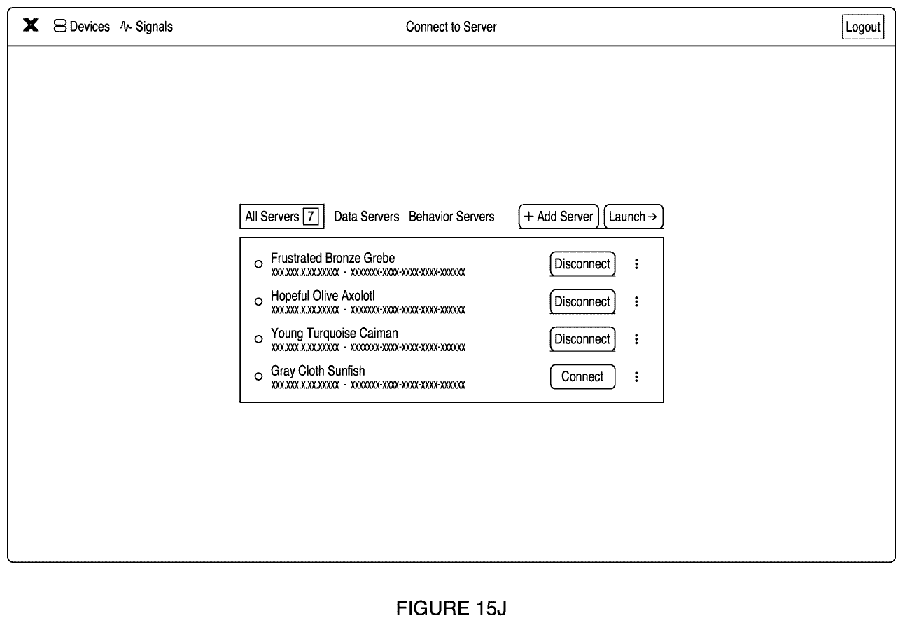

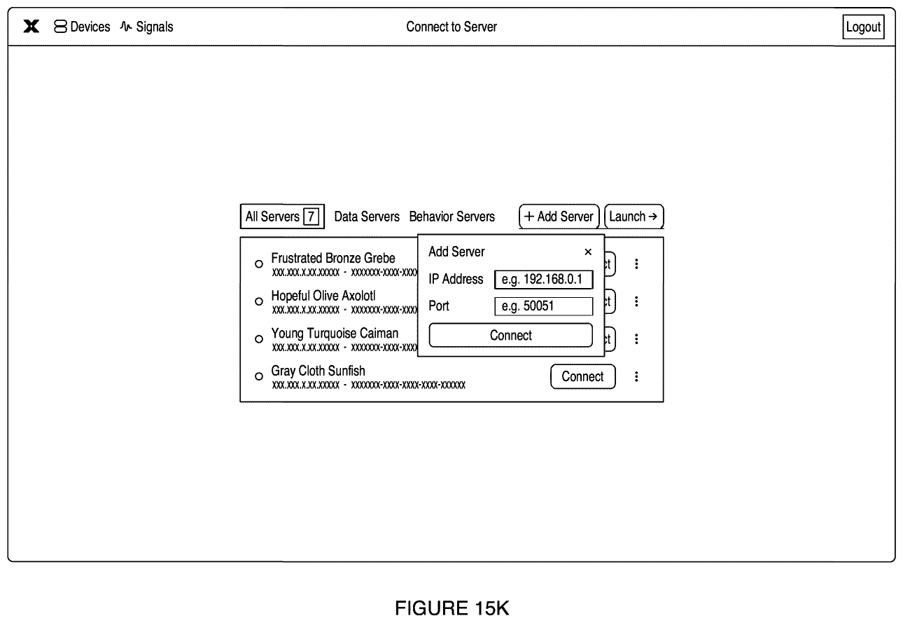

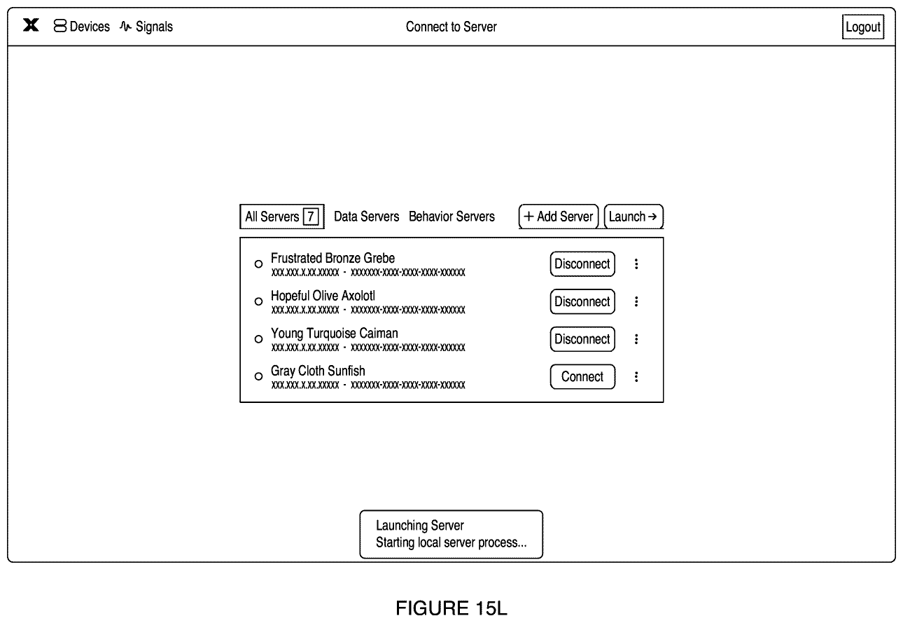

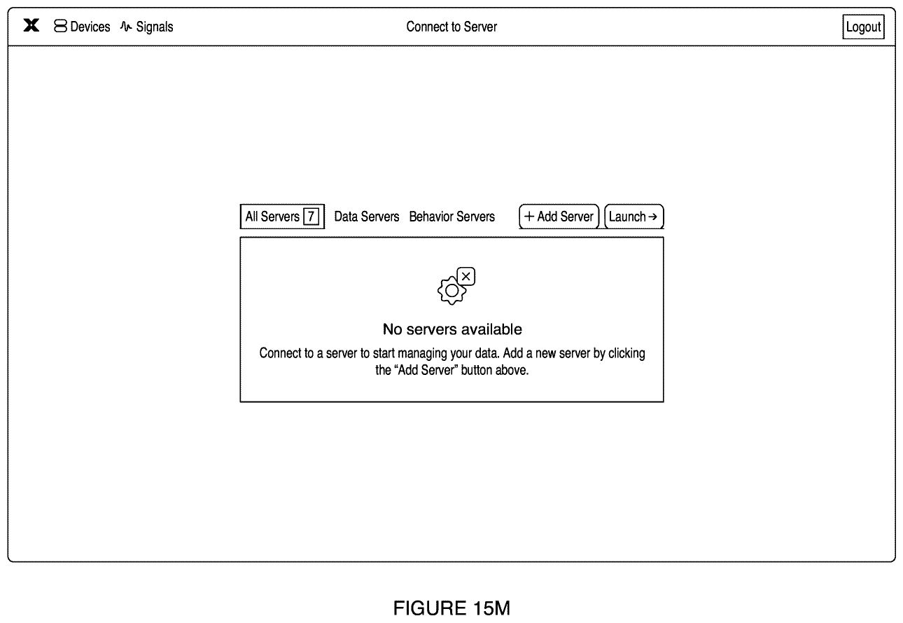

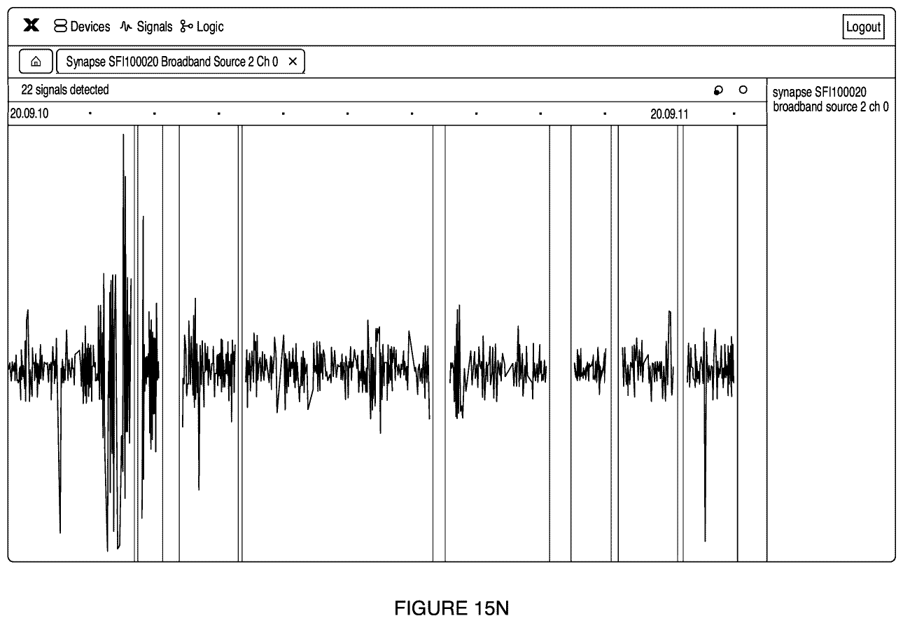

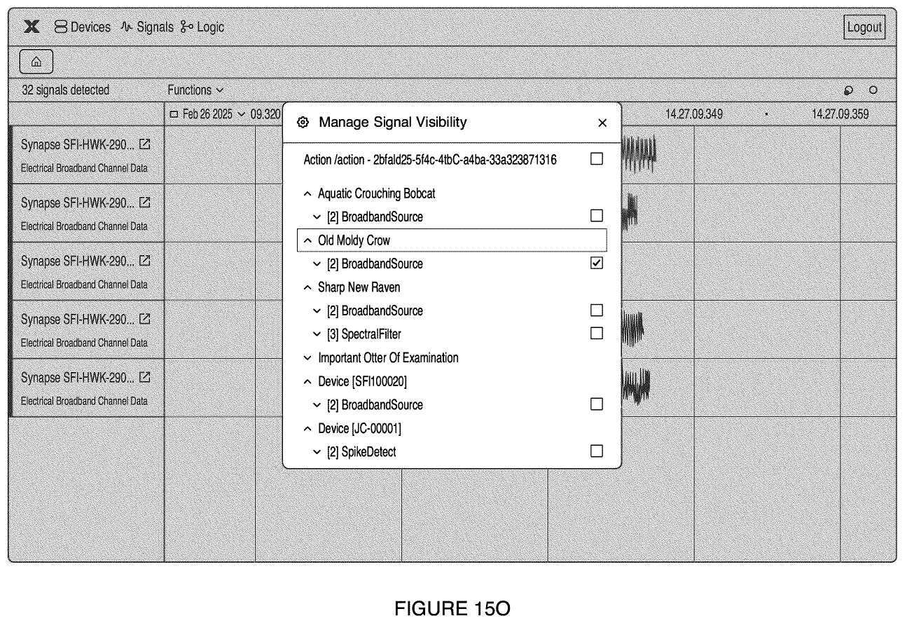

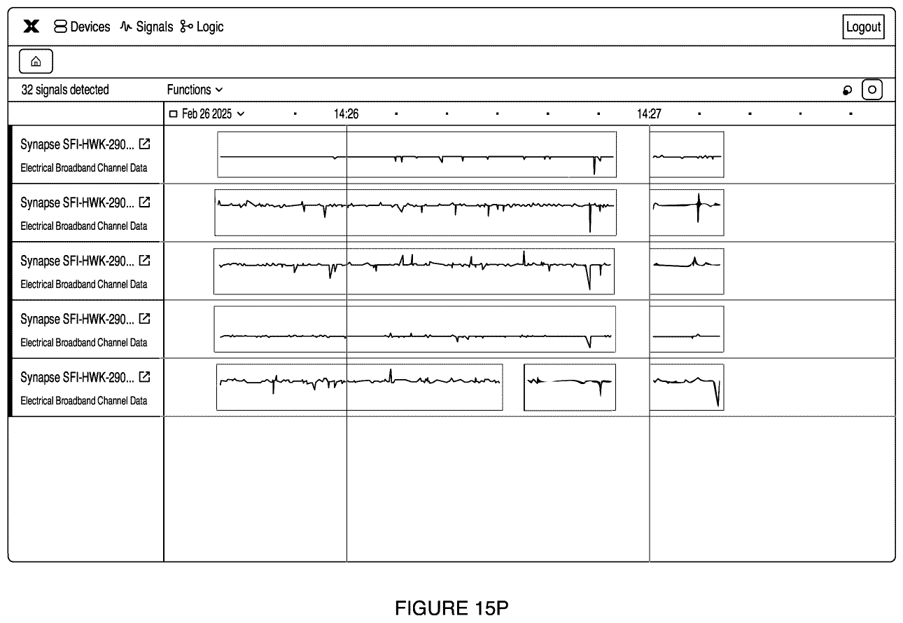

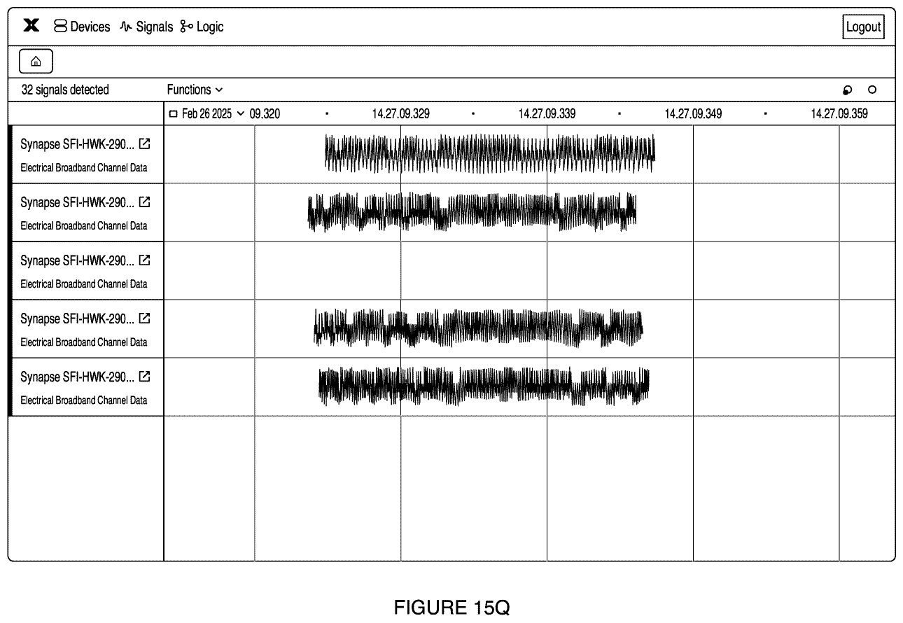

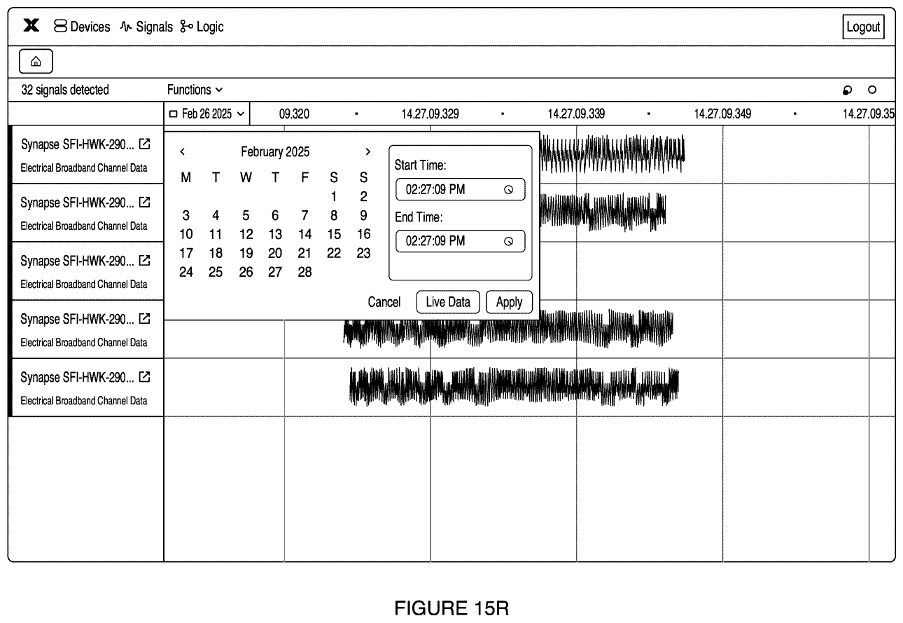

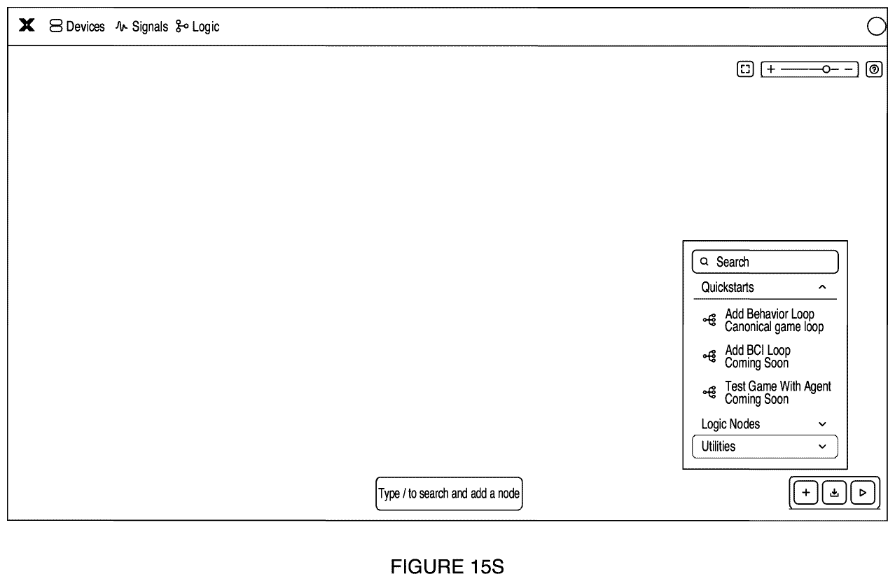

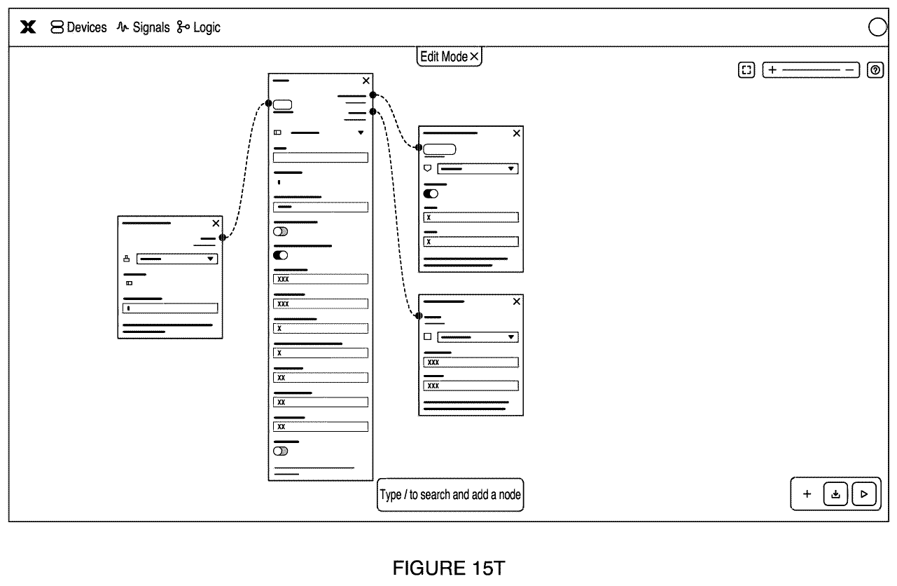

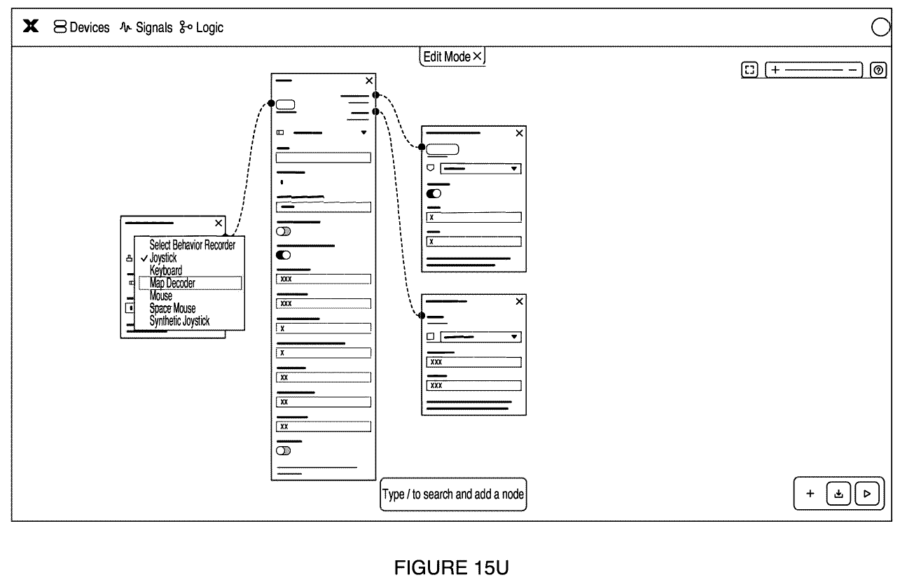

The system can optionally include one or more user interfaces. Examples of a user interface are shown in FIG. 4A, FIG. 4B, FIG. 4C, FIG. 8A, FIG. 8B, and FIGS. 15A-15U. The user interface can be local to the interface device 20 or remote from the interface device 20. The user interface can receive one or more inputs (e.g., signal pipeline parameters, stimulus signals, etc.), display one or more outputs (e.g., processed or unprocessed neural signals, stimulus signals, etc.), display the signal pipeline and/or components thereof (e.g., the arrangement of nodes in the signal pipeline), and/or otherwise function. Inputs can be received from a user, from the interface device 20, from an external system (e.g., an external processing system), and/or from any other system. In specific examples, inputs can be received via a file upload, via button presses, via text input, and/or via any other input action. In a first example, the user interface can include a display for visualizing and/or configuring the signal pipeline. In a specific example, the user interface can receive signal pipeline parameters from a user to configure the signal pipeline. In a second example, the user interface can include a display for visualizing: processed or unprocessed neural signals, stimulus signals, telemetry information, power information, detected external devices (e.g., probes), storage information, and/or any other information. In a third example, the user interface can display an output based on a status criterion (e.g., a predetermined criterion, a criterion defined by a user input, etc.). In a specific example, the user interface can display an output based on the set of neural signals and the status criterion. Examples of status criteria include: a time threshold for changing cell media, a neural spike detection criterion, and/or any other criterion. In a specific example, executing the signal pipeline can include processing the set of neural signals to detect a set of neural spikes according to processing parameters (e.g., received as user inputs), and the status criterion can include a neural spike detection criterion. The user interface can optionally include an LED ring located on the interface device 20. In a first illustrative example, when a time elapsed is greater than the time threshold for changing cell media, the LED ring can change color, turn on, and/or otherwise display an alert. In a second illustrative example, when neural spikes are detected (e.g., via a processing node, as described below), the LED ring can change color, turn on, and/or otherwise display an alert.

However, the user interface can be otherwise configured.

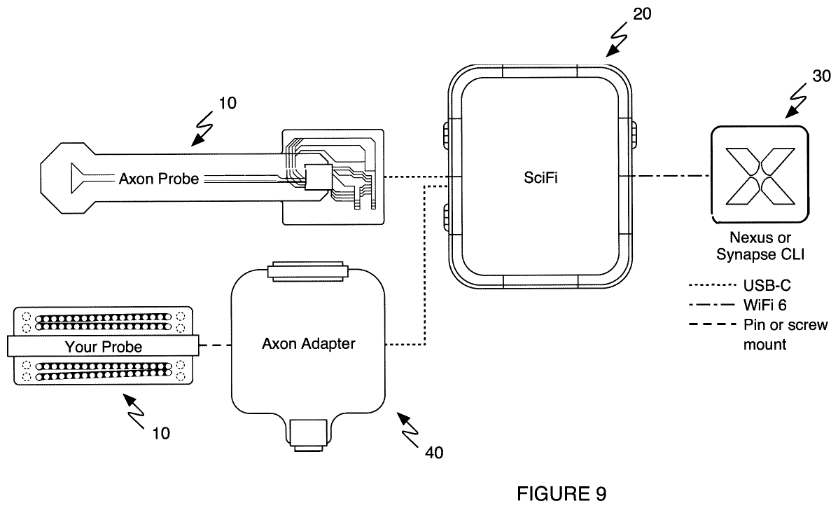

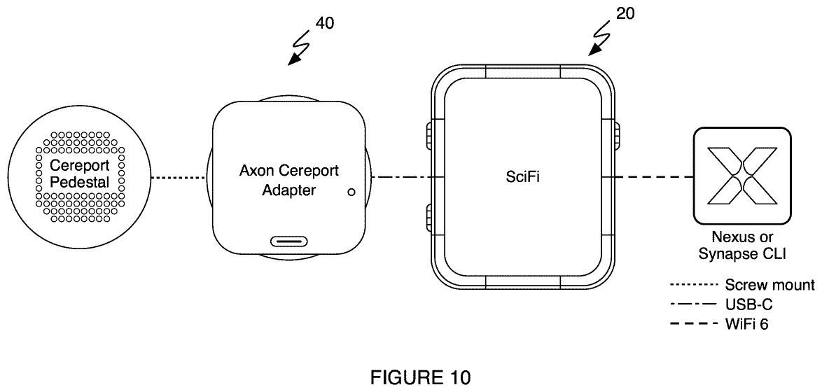

The system can optionally include an adapter 40, which functions to enable the interface device 20 to interface with passive probes (e.g., probes that do not contain all or a portion of an integrated circuit). Examples are shown in FIG. 9 and FIG. 10. The adapter can connect to: the interface device 20, one or more probes 10, and/or one or more supplementary devices.

The adapter 40 can optionally include an integrated circuit (e.g., an integrated circuit as described above). Examples are shown in FIG. 13A and FIG. 13B. The integrated circuit can optionally include one or more of: a set of electrode inputs, routing resources (e.g., a set of switches), a differential amplifier, an analog memory component, an analog-to-digital converter (ADC) component, optional capacitors, a set of general-purpose input/outputs (GPIOs), and/or any other integrated circuit components. In a specific example, the integrated circuit can include an FPGA.

For example, the integrated circuit in the adapter 40 can include: a set of inputs connected to the set of electrodes of the neural probe, a set of switches (e.g., programmable switches), and a set of recording channels (e.g., differential recording channels). In a specific example, each recording channel can include a differential amplifier (e.g., LNA) configured to record a differential signal. In an example, the set of switches can connect the set of inputs to the set of recording channels, where each recording channel is configured to record a differential signal from two inputs (e.g., connected to a recording electrode and a reference electrode).

The adapter 40 can optionally interface with one or more supplementary devices, wherein the adapter 40 can record from the supplementary device and/or transmit to the supplementary device. In a first example, the adapter 40 can record data (e.g., external event occurrence and/or timing, experiment information, measurements, etc.) from the supplementary device. In a second example, the adapter 40 can output a supplementary signal (e.g., stimulus signal) to the stimulation device. In an example, the integrated circuit on the adapter 40 can include GPIOs to interface with the supplementary device.

The adapter 40 can optionally synchronize data recording between the supplementary device and the probe 10. In a first example, the adapter 40 can receive a supplementary signal from the supplementary device synchronously, with a neural signal received from the probe 10. In a specific example, receiving the supplementary signal from the supplementary device synchronously with a neural signal received from the probe 10 can include receiving the supplementary signal and the neural signal within the same time window, where the time window can be less than 0.5 ms, less than 1 ms, less than 10 ms, less than 50 ms, and/or less than 100 ms. In a specific example, receiving the supplementary signal from the supplementary device synchronously with a neural signal received from the probe 10 can include recording the data received from the supplementary device in the same data packet (e.g., data frame) as the neural signal received from the probe 10. In a second example, the adapter 40 can transmit a supplementary signal (e.g., stimulus signal) to the supplementary device synchronously with transmitting a stimulus signal (e.g., electrical signal and/or optical signal) to the probe 10.

The adapter 40 can optionally include systems as described in U.S. application Ser. No. 19/097,738 filed 1 Apr. 2025 and/or U.S. application Ser. No. 19/097,539 filed 1 Apr. 2025, each of which is incorporated in its entirety by this reference.

However, the adapter 40 can be otherwise configured.

System elements (e.g., probe 10, the interface 20, etc.) can optionally communicate and/or be otherwise controlled using an application programming interface (API). In variants, the API can prescribe components and/or other parameters of a signal pipeline (e.g., as described below), prescribe communication protocols, and/or otherwise configure interactions between system elements.

However, the system can be otherwise configured.

### 5. Method

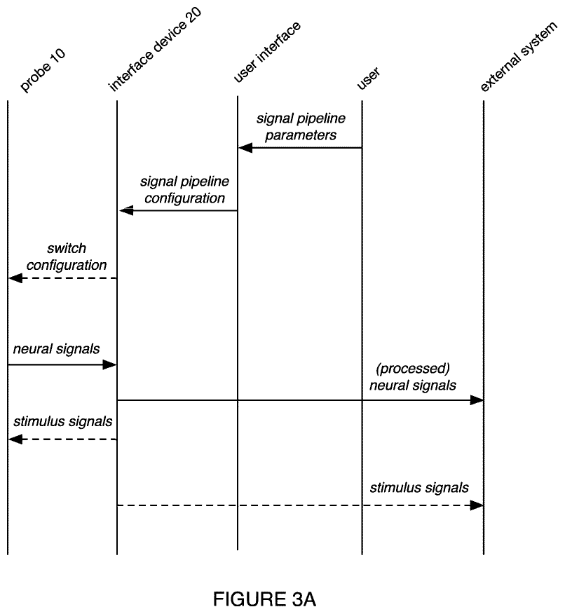

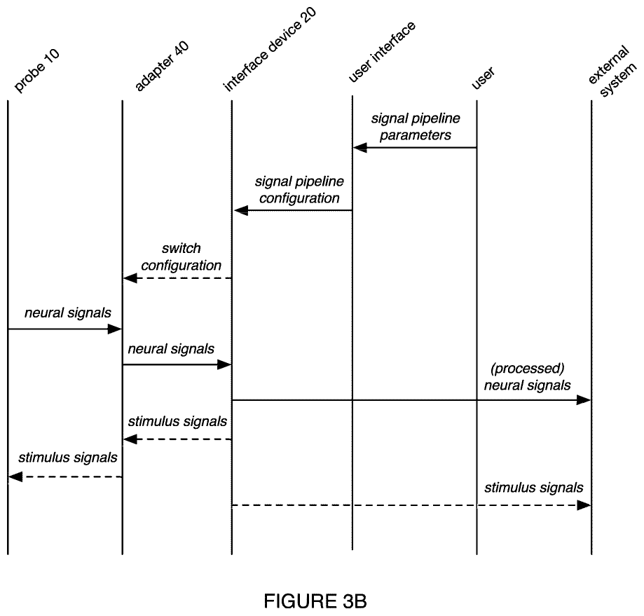

As shown in FIG. 2, the method can include: configuring a signal pipeline S100 and executing the signal pipeline S200. However, the method can additionally or alternatively include any other suitable steps. Specific examples of the method are shown in FIG. 3A and FIG. 3B.

All or portions of the method can be performed by or using: one or more components of the system, a computing system, a database (e.g., a system database, a third-party database, etc.), a user interface, a user, and/or by any other suitable system. All or portions of the method can be performed in real time (e.g., responsive to a request, continuously, etc.), iteratively, concurrently, asynchronously, periodically, and/or at any other suitable time. All or portions of the method can be performed automatically, manually, semi-automatically, and/or otherwise performed.

The method can optionally use one or more models, including a stimulus model, a data processing model, a user-defined model (e.g., user-defined algorithm), and/or any other model. The models can use classical or traditional approaches, machine learning approaches, and/or other approaches. The models can include regression (e.g., linear regression, non-linear regression, logistic regression, etc.), decision tree, LSA, clustering, association rules, dimensionality reduction (e.g., PCA, t-SNE, LDA, etc.), neural networks (e.g., CNN, DNN, CAN, LSTM, RNN, encoders, decoders, deep learning models, transformers, etc.), ensemble methods, optimization methods, classification, rules, heuristics, equations (e.g., weighted equations, etc.), selection (e.g., from a library), regularization methods (e.g., ridge regression), Bayesian methods (e.g., Naive Bayes, Markov, etc.), instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministics, genetic programs, and/or any other suitable architecture. The models can include (e.g., be constructed using) a set of input layers, output layers, and hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); a set of self-attention layers; and/or have any other suitable architecture. The models can include less than 10, tens, hundreds, thousands, tens of thousands, hundreds of thousands, and/or any other number of parameters (e.g., weights, biases, etc.). The models can extract data features (e.g., feature values, feature vectors, high-dimensional features, embeddings in a high-dimensional space with hundreds or thousands of dimensions, human-unintelligible features, etc.) from the input data, and determine the output based on the extracted features. However, the models can otherwise determine the output based on the input data.

Models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The models can be learned or trained on: labeled data (e.g., data labeled with the target label), unlabeled data, positive training sets (e.g., a set of data with true positive labels, negative training sets (e.g., a set of data with true negative labels), and/or any other suitable set of data.

Any model can optionally be validated, verified, reinforced, calibrated, or otherwise updated based on newly received, up-to-date measurements; past measurements recorded during the operating session; historic measurements recorded during past operating sessions; or be updated based on any other suitable data.

Any model can optionally be run or updated: once; at a predetermined frequency; every time the method is performed; every time an unanticipated measurement value is received; or at any other suitable frequency. Any model can optionally be run or updated: in response to determination of an actual result differing from an expected result; or at any other suitable frequency. Any model can optionally be run or updated concurrently with one or more other models, serially, at varying frequencies, or at any other suitable time.

Configuring a signal pipeline S100 functions to prescribe parameters of the signal pipeline based on one or more user inputs. In an example, the signal pipeline (e.g., a signal chain) can be a sequence of nodes prescribing actions to be executed by the interface device 20, by the adapter 40, and/or by the probe 10. In an illustrative example, the signal pipeline can prescribe data events in a brain computer interface experiment.

The signal pipeline can be configured based on a set of signal pipeline parameters (e.g., where the signal pipeline parameters can be user inputs). Specific examples of signal pipeline parameters include: nodes (e.g., node type, node parameters, etc.), node arrangement (e.g., node sequence, node connections, etc.), recording parameters (e.g., neural signal sampling rate), stimulus parameters (e.g., framerate, intensity, pattern, type, etc.), probe specifications, a user-defined algorithm, and/or any other parameters. For example, a signal pipeline can include a set of nodes (e.g., where each node is defined by a set of node parameters) connected according to a node arrangement (e.g., the arrangement of the set of nodes). Examples of probe specifications include: number of electrodes, electrode arrangement (e.g., recording electrode identifiers, reference electrode identifiers, etc.), number of LEDs, LED arrangement, reference mode for the probe, and/or any other probe specifications. For example, the signal pipeline parameters can include: a set of nodes, an arrangement of the set of nodes, and/or a set of parameters for each node (e.g., recording parameters, processing parameters, stimulation parameters, data transfer parameters, user-defined parameters, etc.). The signal pipeline parameters can be: predetermined (e.g., predetermined by the API), automatically determined, manually determined (e.g., determined based on one or more user inputs, received as user inputs, etc.), determined based on a set of heuristics, determined using a model, and/or otherwise determined. In an example, a user can manually input signal pipeline parameters via the user interface.

The signal pipeline can include one or more nodes arranged in a sequence. Nodes can be arranged in parallel and/or in series. Examples of nodes in a signal pipeline include: recording nodes, processing nodes (e.g., data processing nodes), stimulus generation nodes, stimulation nodes, data transfer nodes, user-defined nodes, and/or any other node type. Examples are shown in FIG. 4A, FIG. 4B, and FIG. 4C.

In a first example, a recording node can be associated with (e.g., defined by) a set of recording parameters. Specific examples of recording parameters can include: neural signal sampling rate, packet size (e.g., number of bits to represent a measurement), gain, a low cutoff frequency (e.g., to attenuate the amplitude of neural signals below the low cutoff frequency), high cutoff frequency (e.g., to attenuate the amplitude of neural signals below the low cutoff frequency), probe specifications (e.g., an electrode arrangement, including a recording electrode identifier and a reference electrode identifier for each recording channel), recording mode, any signal pipeline parameters, and/or any other parameters defining recording from a probe 10 (e.g., directly and/or via an adapter). In a second example, a processing node can be associated with (e.g., defined by) a set of processing parameters. Specific examples of processing parameters can include: thresholds (e.g., spike detection voltage thresholds), bin time (e.g., the length of time in ms before the spike count resets), filter parameters (e.g., filter thresholds), user-defined algorithm, probe specifications, any signal pipeline parameters, and/or any other parameters defining processing data. In a third example, a stimulus generation node and/or a stimulation node can be associated with (e.g., defined by) a set of stimulation parameters. Specific examples of stimulation parameters can include: stimulus signal type (e.g., optical stimulus, electrical stimulus, etc.), stimulus device identifier (e.g., the probe 10, a stimulation device, etc.), probe specifications (e.g., electrode identifiers for electrical stimulation; LED identifiers for optical stimulation; etc.), stimulus model, stimulus signal pattern (e.g., predefined, determined based on an input signal received from the probe 10 and/or a supplementary device, etc.), stimulus timing parameters, framerate, intensity, any signal pipeline parameters, and/or any other parameters defining a stimulus signal. In a fourth example, a data transfer node can be associated with (e.g., defined by) a set of data transfer parameters. Specific examples of data transfer parameters can include: output framerate, output device identifier (e.g., remote processing system, supplementary device, etc.), any signal pipeline parameters, and/or any other parameters defining data transfer. In a fifth example, a user-defined node can be associated with (e.g., defined by) a set of user-defined parameters. Specific examples of user-defined parameters can include: user-defined algorithm (e.g., parameters defining a user-defined algorithm), processing parameters, any signal pipeline parameters, and/or any other parameters defined by a user.

However, the signal pipeline can be otherwise configured.

Executing the signal pipeline S200 functions to execute each node of the signal pipeline. In an example, the interface device 20 can receive the signal pipeline from the processing system 30, and execute each node of the signal pipeline.

The API can optionally include one or more commands to start and/or stop execution of the signal pipeline. For example, the interface device 20 can execute each node of the signal pipeline based on the signal pipeline parameters. In a first specific example, the interface device 20 can directly execute a node. In a second specific example, the interface device 20 can control the probe 10, the adapter 40, and/or any other external device to execute a node. Nodes can be executed sequentially (e.g., in series), in parallel, and/or in any other sequence. The signal pipeline and/or any node therein can be executed one or more times. The signal pipeline and/or any node therein can optionally be executed iteratively. Nodes of the signal pipeline are preferably executed in real time, but can alternatively be executed asynchronously. For example, the signal pipeline can be executed in real time in a brain-computer interface experiment (e.g., recording neural signals, generating stimulus signals based on the neural signals, and delivering the stimulus signals in real time).

In a first example, when executing a recording node, the interface device 20 can collect neural signals from the probe 10 (e.g., using a defined data transport protocol). The neural signals can be collected directly from the probe 10 (e.g., an example is shown in FIG. 3A) and/or indirectly from the probe 10, via the adapter 40 (e.g., an example is shown in FIG. 3A). For example, executing the recording node can include controlling the probe 10 and/or the adapter 40 to: record a set of neural signals from a set of recording channels (e.g., on the probe 10 and/or the adapter 40), and optionally transmit the set of neural signals to the interface device 20. In a first specific example, a recording node can be an electrical broadband node, wherein the probe 10 and/or the adapter 40 is controlled to output broadband electrophysiology data (neural signals, from electrodes on the probe 10) to the interface device 20 as a collection of channels with the same data rate. In a second specific example, a recording node can be an optical broadband node, wherein the probe 10 and/or the adapter 40 is controlled to output broadband optical data from photodiodes to the interface device 20 as a collection of channels with the same data rate. The data transport protocol can define the sampling rate and packet size for transporting packets of data from the probe 10 and/or the adapter 40 to the interface device 20. Each packet can include data (e.g., a single value) from each recording electrode of the probe 10 corresponding to a given timestamp. In a specific example, when the probe 10 is configured in a differential mode (e.g., as described below), the packet can include data (e.g., a single value) from each pair of electrodes. The packet size can optionally be determined based on the number of electrodes (e.g., the number of electrodes designated as recording electrodes) in the probe 10. The sampling rate is preferably at least 10,000 Hz (e.g., at least 15,000 Hz, at least 20,000 Hz, at least 30,000 Hz), but can alternatively be less than 10,000 Hz.

In a second example, when executing a processing node (e.g., data processing node), the interface device 20 can process the recorded neural signals. Examples of processing include: spike detection, filtering, downsampling, normalizing, extracting signal parameters, transforming, aggregating, statistical analysis, fitting, denoising, applying a processing model, applying a user-defined model, a combination thereof, and/or otherwise processing a signal. In a first example, a processing node can be a spike detection node, wherein the interface device 20 detects action potentials (neural spikes) using a spike thresholding algorithm and outputs spike train data. In an illustrative example, executing the processing node can include counting the number of times the absolute value of the inputted data (e.g., neural signal) crosses a threshold within a set time period (e.g., assumes a refractory period of ˜1 ms between spikes). In a specific example, user inputs can include processing parameters, wherein executing the signal pipeline can include processing the set of neural signals to detect a set of neural spikes according to the processing parameters. In a specific example, the system can include a remote system configured to receive the user inputs, wherein the interface device 20 is communicatively connected to the remote system. In a second example, a processing node can be a spectral filter node, wherein the interface device 20 applies a basic frequency-domain filter (e.g., high-pass, low-pass, band-pass, etc.) to a set of neural signals.

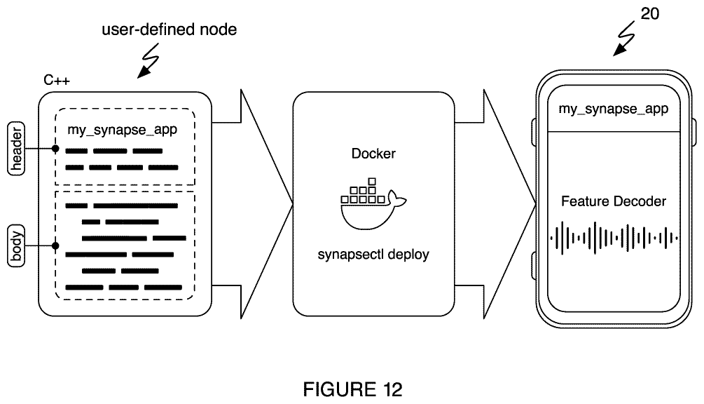

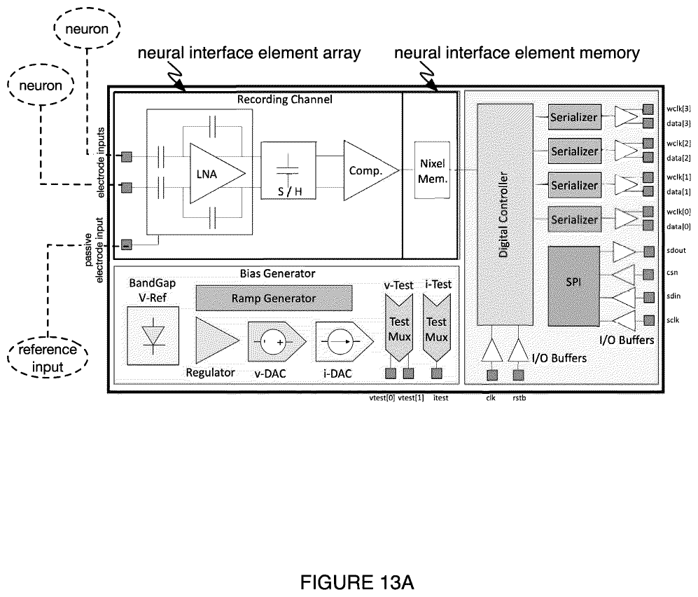

In a third example, when executing a user-defined node, the interface device 20 can execute one or more actions as defined by the user-defined node. For example, executing the user-defined node can include processing data (e.g., processed and/or unprocessed neural signals) based on (e.g., according to) a user-defined algorithm. In a specific example, the user-defined algorithm (e.g., a custom neural processing algorithm) can decode neural signals. In a specific example, the set of user inputs can include the user-defined algorithm. In another specific example, a set of parameters for the user-defined node can include the user-defined algorithm. In variants, this can enable real-time data processing with minimal latency. An example is shown in FIG. 12.

In a fourth example, when executing a stimulus generation node, the interface device 20 can determine one or more stimulus signals. The stimulus signal can be a signal for an electrical stimulus, optical stimulus (e.g., light signal), visual stimulus (e.g., text, images, video, cursor location, etc.; displayed at a user interface), audio stimulus, an action, any other feedback, and/or any other stimulus. In a first specific example, stimulus signals can be determined based on received data (e.g., received via a data transfer node). For example, the stimulus signal can be the received data. In a second specific example, stimulus signals can be determined based on the neural signals (e.g., processed or unprocessed neural signals). For example, a stimulus model can be used to determine stimulus signals.

In a fifth example, when executing a stimulation node, the interface device 20 can control the probe 10 and/or adapter 40 to apply the stimulus signals (e.g., electrical signals and/or optical signals) via electrodes and/or LEDs. Examples are shown in FIG. 3A and FIG. 3B. In a first specific example, the stimulation node can be an electrical stimulation node, wherein the interface device 20 transmits stimulus signals (e.g., generated via the stimulus generation node) to the probe 10 and/or adapter 40 to stimulate neurons via electrodes. In a second specific example, the stimulation node can be an optical stimulation node, wherein the interface device 20 transmits stimulus signals to the probe 10 and/or adapter 40 to stimulate neurons via LEDs. In a specific example, executing the simulation node can include controlling the probe 10 to drive a set of LEDs on the probe 10 (e.g., based on the set of neural signals and/or stimulation parameters).

In a sixth example, when executing a data transfer node, the interface device 20 can transmit and/or receive data (e.g., processed or unprocessed neural signals, stimulus signals, any other data) from an external system (e.g., a processing system 30, the probe 10, etc.). Examples are shown in FIG. 3A and FIG. 3B. In a first specific example, a data transfer node can be a data stream out node, wherein a data stream can be transferred from the interface device 20 to a host computer. In a second specific example, a data transfer node can be a data stream in node, wherein a data stream can be transferred from a host computer to the interface device 20.

Executing the signal pipeline S200 can optionally include configuring the probe 10. For example, the probe 10 can be configured prior to and/or as part of a recording node. The probe 10 can optionally validate this configuration and/or return an error message if the configuration is not valid. The probe 10 can be configured based on predetermined specifications, automatically, manually (e.g., based on user inputs), and/or otherwise determined. In a specific example, the probe 10 can be configured based on a set of user inputs (e.g., a configuration file containing a set of user inputs). The user inputs can include probe specifications. Examples of probe specifications include: reference electrode identifiers, recording electrode identifiers, number of electrodes, number of LEDs, arrangement of electrodes, arrangement of LEDs, LED wavelength, sampling frequency, and/or any other probe information.

Configuring the probe 10 can optionally include controlling a set of switches (e.g., programmable switches) on the probe 10 and/or the adapter 40 based on the probe specifications (e.g., based on the electrode arrangement). For example, the set of switches can be controlled to connect two electrodes of the set of electrodes to each recording channel of a set of recording channels in the probe 10 and/or adapter 40. In an example, the electrode arrangement can include a recording electrode identifier and a reference electrode identifier assigned to each recording channel of the set of recording channels. In a specific example, the set of switches can be controlled to connect the set of recording channels to the corresponding electrodes (e.g., each recording channel is connected to a pair of electrodes corresponding to the assigned recording electrode identifier and a reference electrode identifier). In an illustrative example, when a recording electrode identifier corresponding to electrode A on the probe 10 and a reference electrode identifier corresponding to electrode B on the probe 10 are assigned to a given recording channel, the set of switches can be programmed to connect electrode A and electrode B to the given recording channel. In a first example, for an interface device connected to a probe, executing the signal pipeline can include: controlling the set of programmable switches on the probe 10 based on the probe specifications to connect two electrodes of the set of electrodes on the probe 10 to each recording channel of the set of recording channels on the probe 10; and controlling the probe 10 to: record a set of neural signals from the set of recording channels and transmit the set of neural signals to the interface device 20. In a second example, for an interface device connected to a probe via an adapter 40, executing the signal pipeline can include: controlling the set of programmable switches on the adapter based on the probe specifications to connect two electrodes of the set of electrodes on the probe 10 to each recording channel of the set of recording channels on the adapter 40; and controlling the adapter to: record a set of neural signals from the set of recording channels and transmit the set of neural signals to the interface device 20.

Configuring the probe 10 can optionally include determining a reference mode for the probe 10. In a first embodiment, the probe 10 can be configured in a global reference mode. For example, the set of electrodes of the probe 10 can include a set of recording electrodes and a global reference electrode (e.g., a single global reference electrode). In this example, for each electrode in the set of recording electrodes, a recorded neural signal is referenced to the global reference electrode. In a second embodiment, the probe 10 can be configured in a differential reference mode. For example, the set of electrodes of the probe 10 can include pairs of electrodes, wherein the probe 10 can record a differential signal from each pair of electrodes (e.g., wherein, at different points in time, one electrode in the pair of electrodes acts as a reference electrode and the other electrode in the pair acts as a recording electrode). In variants, this differential mode assumes that a neural pulse will come from only one electrode of a pair of electrodes at a given time, while the other idle electrode can act as a reference. In a specific example, the polarity of a detected neural signal from a pair of electrodes can indicate which electrode of a pair received the neural signal (e.g., which electrode of the pair acted as the recording electrode). In an illustrative example, one electrode of each pair corresponds to a positive signal while the other electrode corresponds to a negative signal.

The reference mode can optionally be determined by assigning (e.g., based on a user input) each electrode a reference electrode identifier (e.g., reference electrode ID) and/or a recording electrode identifier (e.g., recording electrode ID). In a first embodiment, in the global reference mode, a single electrode of the probe 10 can be assigned a reference electrode identifier and remaining electrodes of the probe 10 can be assigned a recording electrode identifier. In a specific example, in the global reference mode, the reference electrode identifier can be the same for each recording channel of the set of recording channels. In a second embodiment, in the differential reference mode, the electrodes of the probe 10 can be grouped into pairs, wherein one electrode of each pair is assigned a reference electrode identifier and the other electrode is assigned a recording electrode identifier. In an example, in the differential reference mode, the reference electrode identifier can be different for each recording channel of the set of recording channels. In a specific example, when the sign of a neural signal recorded from a recording channel is negative, the neural signal corresponds to the reference electrode identifier (e.g., the neural signal originated at a neuron interfacing with the electrode corresponding to the reference electrode identifier); when the sign of a neural signal recorded from a recording channel is positive, the neural signal corresponds to the recording electrode (e.g., the neural signal originated at a neuron interfacing with the electrode corresponding to the recording electrode identifier). In an illustrative example, for a pair of electrodes, Electrode A and Electrode B, a user can assign Electrode A a recording electrode identifier and assign Electrode B a reference electrode identifier, wherein the sign of a differential signal recorded from Electrode A and Electrode B (e.g., the signal from Electrode A referenced to Electrode B) can indicate the origin of the neural signal. For example, when a neural signal is received at Electrode A, the differential signal is positive, and when a neural signal is received at Electrode B, the differential signal is negative. In a third specific example, in the differential reference mode, the electrodes of the probe 10 can be grouped into pairs, wherein each electrode of each pair is assigned both a reference electrode identifier and a recording electrode identifier.

In an example, a spatial map of the recording electrodes and the reference electrodes can be inferred based on the recording electrode identifiers and the reference electrode identifiers (e.g., a configuration file containing the recording electrode identifier s and the reference electrode identifiers), wherein the data pipeline can preserve the association recorded signals and electrode identifiers. In a specific example, when the reference mode is a differential mode and a differential signal is recorded from a pair of electrodes, the signal can be mapped to the physical electrode where the signal originated based on the spatial map and the sign of the differential signal.

However, the probe 10 can be otherwise configured.

However, the signal pipeline can be otherwise executed.

### 6. Specific Examples

In a specific example, the interface device 20 (e.g., SciFi) is a high bandwidth neural recording headstage. The interface device 20 can enable a user to perform complex precision experiments with ease and flexibility. The interface device 20 can optionally support neural stimulation and high-bandwidth data acquisition over WiFi 6, featuring UDP multicast low-latency streaming, service autodiscovery, and/or plug-and-play functionality. The interface device 20 can optionally feature WiFi 6 (2×2 MIMO) wireless connectivity for ultra-low latency, with flexible antenna configurations (both internal and external). The interface device 20 can optionally be ruggedized to meet harsh operating conditions and can be plug and play compatible with probes (e.g., Science Axon Probes) and an API (e . . . g, the Synapse API).

In specific examples, the interface device 20 can optionally include one or more of the following features: (a) WiFi 6 connectivity for minimal latency and connection speed of up to 2.4 Gbps; (b) High contrast OLED display for detailed status and experiment information; (c) High visibility RGB light ring for status indication provides information about the device to users who may be some distance away (e.g., where the light can optionally be customizable by the user for their experiment goals); (d) Internal WiFi antennas included, can be configured to use external antennas; (e) 1400 mAh battery (e.g., 2-4 hour battery life in active use); (f) 128 GB of internal high speed storage available for saving recordings on-device; (g) Powered by Qualcomm Dragonwing Q6; (h) Status indication through LED ring and AMOLED display panel; (i) USB Type-C® port for connectivity.

In a specific example, an up/down button on the interface device 20 can navigate between options on the display of the interface device 20 (e.g., an AMOLED screen). In another specific example, holding a power/select button on the interface device 20 can turn on and/or off the device. In another specific example, short presses of a power/select button on the interface device 20 can serve as a select key. In another specific example, an LED status ring on the interface device 20 can express a range of intuitive status information and/or can be customized to fit experiment goals (e.g., timer when cell media needs to be swapped, spikes being detected, etc.). An example is shown in FIG. 8A.

In a specific example, to record from a probe, the recording configuration can be specified using a synapse JSON file. For example, a configuration file specifies the nodes in the signal chain, as well as how they are connected. For example, a configuration file can define which channels are being recorded from, the bit depth and sample rate of the recording, and/or the high- and low-pass filter corner frequencies. In a specific example, “synapsectl read $DEVICE_IP--config $PATH_TO_CONFIG_FILE.json” can be used to start recording. The recording can optionally run indefinitely. A recording can optionally be ended by pressing CTRL+C in the terminal window. A recording session can optionally be supplemented with one or more of the following functions: (a) “--duration $TIME_IN_SECONDS” to specify recording duration; (b) “--plot” to enable live plotting GUI; (c) “--bin true” to save data to binary format; (d) “--output $OUTPUT_FILE_NAME” to specify output filename; and/or (e) “--overwrite” to overwrite existing data if filename is identical.

A specific example of a GUI for the interface device 20 can display live plotting data. In a specific example, the GUI can have two views that can be toggled with the top navigation bar: All Channels and Zoomed Channel. In a specific example, the left side of the window contains the configuration settings (e.g., which channel is displayed in the zoomed view; the bounds of the y-axis in the zoomed view; distance between traces in the all channels view (signal separation); etc.). In a specific example, the x axis can be rescaled by right-clicking on the graph window.

In a specific example, by default, the read command will save a folder with the data and the runtime configuration. In a specific example, a user can point the synapsectl plot command to a directory to plot the data. In an example, a user can change the selected channel in the bottom window with the dropdown menu in the upper left corner. In a specific example, a user can scroll across plots with left click and drag and/or change x- and y-axis scales with right click and drag. In a specific example, a user can right click on a window to manually adjust the plot settings.

In a specific example, an impedance measurement can be requested from a probe 10 by constructing and formatting an “Impedance Query” message to send to the interface device 20 (e.g., including a list of electrode identifiers). In a specific example, after the measurement finishes, an output is generated with a status (e.g., typically blank) and a list of each electrode measurement measured by magnitude (Ohms) and phase (degrees).

In a specific example, the interface device 20 can interface with a recording peripheral that can implement the Synapse Electrical Broadband node. In a specific example the following ElectricalBroadbandConfig parameters can be used:

```
  bit_width: 12
  sample_rate: 4000, 8000, 16000, 20000, 25000, 32000
  gain: 200
  low_cutoff_hz: 57, 66, 77, 92, 114, 154, 234, 457 (defaults 92 if field is
empty)
  high_cutoff_hz: 1445, 1862, 2630, 4365, 13489 (defaults 13489 if field is
empty)
  channels: valid electrode_id and reference_id depend on the desired
reference mode
```

In a first specific example, in Differential Mode, a user can select pairs of electrodes according to the following rules: electrode_id: Select of set of even numbered electrodes which fall between 0-511, inclusive; reference_id: For a given electrode_id n, reference_id=n+1.

In a second specific example, in Global Reference Mode, the following rules can be used: electrode_id: All even OR all odd values between 0-511, inclusive; reference_id: refer to the following:

electrode_id 0-7: 512
electrode_id 8-127: 513
electrode_id 128-135: 514
electrode_id 136-255: 515
electrode_id 256-263: 516
electrode_id 264-383: 517
electrode_id 384-391: 518
electrode_id 392-511: 519

In a specific example, a virtual recording peripheral can generate test data for benchmarking and debugging the interface device 20. The virtual recording peripheral can optionally implement the Electrical Broadband node. In a specific example, the virtual recording peripheral can attempt to match the bit-rate requested by the supplied config parameters.

A numbered list of specific examples of the technology described herein are provided below. A person of skill in the art will recognize that the scope of the technology is not limited to and/or by these specific examples.

Specific Example 1. A system, comprising: a neural probe interfacing with a set of neurons, the neural probe comprising: a set of electrodes; a set of recording channels; and a set of programmable switches connecting the set of electrodes to the set of recording channels; and an interface device connected to the neural probe, the interface device configured to: receive a set of user inputs comprising neural probe specifications; and execute a signal pipeline, wherein executing the signal pipeline comprises: controlling the set of programmable switches based on the neural probe specifications to connect two electrodes of the set of electrodes to each recording channel of the set of recording channels; and controlling the neural probe to: record a set of neural signals from the set of recording channels and transmit the set of neural signals to the interface device.

Specific Example 2. The system of Specific Example 1, wherein the neural probe specifications comprise an electrode arrangement, the electrode arrangement comprising a recording electrode identifier and a reference electrode identifier for each recording channel of the set of recording channels.

Specific Example 3. The system of Specific Example 2, wherein the reference electrode identifier is different for each recording channel of the set of recording channels.

Specific Example 4. The system of any of Specific Examples 2-3, wherein the reference electrode identifier is the same for each recording channel of the set of recording channels.

Specific Example 5. The system of any of Specific Examples 1-4, further comprising a remote system configured to receive the set of user inputs, wherein the interface device is communicatively connected to the remote system, wherein the set of user inputs further comprises a user-defined algorithm, wherein executing the signal pipeline further comprises processing the set of neural signals according to the user-defined algorithm.

Specific Example 6. The system of any of Specific Examples 1-5, wherein the set of neurons comprise optogenetically modified neurons, the neural probe further comprising a set of LEDs configured to transmit light signals to the set of neurons, wherein an LED in the set of LEDs transmits a light signal to a neuron of interest in the set of neurons, and wherein an electrode in the set of electrodes receives an electrical signal from the neuron of interest, wherein the set of user inputs further comprises stimulation parameters, wherein executing the signal pipeline further comprises: controlling the neural probe to drive the set of LEDs based on the set of neural signals and the stimulation parameters.

Specific Example 7. The system of any of Specific Examples 1-6, wherein the set of user inputs further comprises a status criterion, wherein the interface device further comprises a user interface configured to display an output based on the set of neural signals and the status criterion.

Specific Example 8. The system of Specific Example 7, wherein the set of user inputs further comprises processing parameters, wherein executing the signal pipeline further comprises processing the set of neural signals to detect a set of neural spikes according to the processing parameters, wherein the status criterion comprises a neural spike detection criterion.

Specific Example 9. The system of any of Specific Examples 7-8, wherein the user interface comprises an LED ring.

Specific Example 10. The system of any of Specific Examples 1-9, wherein the neural probe further comprises a cell container arranged above the set of electrodes, wherein the set of neurons are cultured within the cell container.

Specific Example 11. A system, comprising: a neural probe comprising a set of electrodes; an adapter connected to the neural probe, wherein the adapter comprises: a set of inputs connected to the set of electrodes of the neural probe; a set of recording channels; and a set of programmable switches connecting the set of inputs to the set of recording channels; an interface device connected to the adapter, the interface device configured to: receive a set of user inputs comprising neural probe specifications; and execute a signal pipeline, wherein executing the signal pipeline comprises: controlling the set of programmable switches based on the neural probe specifications to connect two electrodes of the set of electrodes to each recording channel of the set of recording channels; and controlling the adapter to: record a set of neural signals from the set of recording channels and transmit the set of neural signals to the interface device.

Specific Example 12. The system of Specific Example 11, wherein the neural probe specifications comprise an electrode arrangement, the electrode arrangement comprising a recording electrode identifier and a reference electrode identifier assigned to each recording channel of the set of recording channels.

Specific Example 13. The system of Specific Example 12, wherein the reference electrode identifier is different for each recording channel of the set of recording channels.

Specific Example 14. The system of Specific Example 13, wherein a first recording electrode identifier and a first reference electrode identifier are assigned to a first recording channel in the set of recording channels, wherein, when a sign of a first neural signal recorded from the first recording channel is negative, the first neural signal corresponds to the reference electrode identifier.

Specific Example 15. The system of any of Specific Examples 11-14, further comprising a remote system configured to receive the set of user inputs, wherein the interface device is communicatively connected to the remote system, wherein the set of user inputs further comprises a user-defined algorithm, wherein executing the signal pipeline further comprises processing the set of neural signals according to the user-defined algorithm.

Specific Example 16. The system of any of Specific Examples 11-15, wherein the set of neurons comprise optogenetically modified neurons, the neural probe further comprising a set of LEDs configured to transmit light signals to the set of neurons, wherein an LED in the set of LEDs transmits a light signal to a neuron of interest in the set of neurons, and wherein an electrode in the set of electrodes receives an electrical signal from the neuron of interest, wherein the set of user inputs further comprises stimulation parameters, wherein executing the signal pipeline further comprises: controlling the neural probe to drive the set of LEDs based on the set of neural signals and the stimulation parameters.

Specific Example 17. The system of any of Specific Examples 11-16, further comprising a remote system configured to receive the set of user inputs, wherein the interface device is communicatively connected to the remote system, wherein the set of user inputs further comprises processing parameters, wherein executing the signal pipeline further comprises processing the set of neural signals to detect a set of neural spikes according to the processing parameters.

Specific Example 18. The system of any of Specific Examples 11-17, further comprising a supplementary device connected to the adapter, wherein the adapter receives a supplementary signal from the supplementary device synchronously with the neural signal received from the neural probe.

Specific Example 19. The system of any of Specific Examples 11-18, further comprising a stimulation device connected to the adapter, wherein the adapter outputs a supplementary signal to the stimulation device.

Specific Example 20. The system of any of Specific Examples 11-19, wherein the neural probe is implanted within a brain or implanted on the surface of a brain.

Specific Example 21. A method, comprising: receiving a set of user inputs from a user, the set of user inputs comprising a set of nodes, an arrangement of the set of nodes, and a set of parameters for each of the set of nodes, wherein the set of nodes comprises a recording node, wherein the set of parameters for the recording node comprises an electrode arrangement, the electrode arrangement comprising a recording electrode identifier and a reference electrode identifier for each recording channel of a set of recording channels in a neural probe; determining a signal pipeline based on the set of user inputs, the signal pipeline comprising the set of nodes connected according to the arrangement of the set of nodes; and executing each node of the signal pipeline using an interface device connected to the neural probe, wherein executing the recording node of the signal pipeline comprises: based on the electrode arrangement, controlling a set of programmable switches connected to a set of electrodes in the neural probe; and recording a set of neural signals from the neural probe.

Specific Example 22. The method of Specific Example 21, wherein the set of nodes further comprises a user-defined node, wherein the set of parameters for the user-defined node comprises a user-defined algorithm, wherein executing the user-defined node comprises processing the set of neural signals according to the user-defined algorithm.

Specific Example 23. The method of any of Specific Examples 21-22, wherein the set of nodes further comprises a stimulus generation node and a stimulation node, wherein executing the stimulus generation node comprises determining a stimulus signal based on the set of neural signals, and wherein executing the stimulation node comprises controlling the neural probe to apply the stimulus signal.

Specific Example 24. The method of Specific Example 23, wherein the stimulus signal comprises a light signal, wherein the neural probe interfaces with a set of optogenetically modified neurons.

Specific Example 25. The method of any of Specific Examples 21-24, wherein the set of programmable switches are located on the neural probe.

Specific Example 26. The method of any of Specific Examples 21-25, wherein the set of programmable switches are located on an adapter connected to the neural probe and the interface device.

Specific Example 27. The method of any of Specific Example 21-26, wherein the neural probe comprises a set of electrodes, wherein controlling the set of programmable switches based on the electrode arrangement comprises controlling the set of programmable switches to connect two electrodes of the set of electrodes to each recording channel of the set of recording channels.

Specific Example 28. The method of Specific Example 27, wherein the reference electrode identifier is different for each recording channel of the set of recording channels.

Specific Example 29. The method of any of Specific Examples 27-28, wherein the reference electrode identifier is the same for each recording channel of the set of recording channels.

Specific Example 30. The method of any of Specific Examples 21-29, wherein the neural probe is implanted within a brain or implanted on the surface of a brain.

Specific Example 31. A system, comprising: a processing system storing computer-readable instructions that, when executed, cause the processing system to: receive a set of user inputs, the set of user inputs comprising a set of nodes, an arrangement of the set of nodes, and a set of parameters for each of the set of nodes, wherein the set of nodes comprises a recording node, wherein the set of parameters for the recording node comprises an electrode arrangement, the electrode arrangement comprising a recording electrode identifier and a reference electrode identifier for each recording channel of a set of recording channels in a neural probe; and determine a signal pipeline based on the set of user inputs, the signal pipeline comprising the set of nodes connected according to the arrangement of the set of nodes; and an interface device communicatively connected to the processing system, wherein the interface device is connected to the neural probe, wherein the interface device configured to: receive the signal pipeline from the processing system; and execute each node of the signal pipeline, wherein executing the recording node of the signal pipeline comprises: controlling a set of programmable switches in the neural probe based on the electrode arrangement; and recording a set of neural signals from the neural probe.

Specific Example 32. The system of Specific Example 31, wherein the set of nodes further comprises a user-defined node, wherein the set of parameters for the user-defined node comprises a user-defined algorithm, wherein executing the user-defined node comprises processing the set of neural signals according to the user-defined algorithm.

Specific Example 33. The system of any of Specific Examples 31-32, wherein the set of nodes further comprises a stimulus generation node and a stimulation node, wherein executing the stimulus generation node comprises determining a stimulus signal based on the set of neural signals, and wherein executing the stimulation node comprises controlling the neural probe to apply the stimulus signal.

Specific Example 34. The system of Specific Example 33, wherein the stimulus signal comprises a light signal, wherein the neural probe interfaces with a set of optogenetically modified neurons.

Specific Example 35. The system of any of Specific Examples 31-34, wherein the reference electrode identifier is different for each recording channel of the set of recording channels.

Specific Example 36. The system of any of Specific Examples 31-35, further comprising a user interface configured to display the arrangement of the set of nodes.

Specific Example 37. The system of any of Specific Examples 31-36, wherein executing each node of the signal pipeline comprises executing each node in series.

Specific Example 38. The system of any of Specific Examples 31-37, further comprising the neural probe, wherein the neural probe comprises: a set of electrodes interfacing with a set of neurons; the set of recording channels; and the set of programmable switches, wherein the set of programmable switches connect the set of electrodes to the set of recording channels.

Specific Example 39. The system of Specific Example 38, wherein controlling the set of programmable switches based on the electrode arrangement comprises controlling the set of programmable switches to connect two electrodes of the set of electrodes to each recording channel of the set of recording channels.

Specific Example 40. The system of any of Specific Examples 38-39, wherein the neural probe further comprises a cell container arranged above the set of electrodes, wherein the set of neurons are cultured within the cell container.

All references cited herein are incorporated by reference in their entirety, except to the extent that the incorporated material is inconsistent with the express disclosure herein, in which case the language in this disclosure controls.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

Optional elements, which can be included in some variants but not others, are indicated in broken line in the figures.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A system, comprising:
a neural probe interfacing with a set of neurons, the neural probe comprising:
a set of electrodes;
a set of recording channels; and
a set of programmable switches connecting the set of electrodes to the set of recording channels; and
an interface device connected to the neural probe, the interface device configured to:
receive a set of user inputs comprising neural probe specifications; and
execute a signal pipeline, wherein executing the signal pipeline comprises:
controlling the set of programmable switches based on the neural probe specifications to connect two electrodes of the set of electrodes to each recording channel of the set of recording channels; and
controlling the neural probe to: record a set of neural signals from the set of recording channels and transmit the set of neural signals to the interface device.

2. The system of claim 1, wherein the neural probe specifications comprise an electrode arrangement, the electrode arrangement comprising a recording electrode identifier and a reference electrode identifier for each recording channel of the set of recording channels.

3. The system of claim 2, wherein the reference electrode identifier is different for each recording channel of the set of recording channels.

4. The system of claim 2, wherein the reference electrode identifier is the same for each recording channel of the set of recording channels.

5. The system of claim 1, further comprising a remote system configured to receive the set of user inputs, wherein the interface device is communicatively connected to the remote system, wherein the set of user inputs further comprises a user-defined algorithm, wherein executing the signal pipeline further comprises processing the set of neural signals according to the user-defined algorithm.

6. The system of claim 1, wherein the set of neurons comprise optogenetically modified neurons, the neural probe further comprising a set of LEDs configured to transmit light signals to the set of neurons, wherein an LED in the set of LEDs transmits a light signal to a neuron of interest in the set of neurons, and wherein an electrode in the set of electrodes receives an electrical signal from the neuron of interest, wherein the set of user inputs further comprises stimulation parameters, wherein executing the signal pipeline further comprises: controlling the neural probe to drive the set of LEDs based on the set of neural signals and the stimulation parameters.

7. The system of claim 1, wherein the set of user inputs further comprises a status criterion, wherein the interface device further comprises a user interface configured to display an output based on the set of neural signals and the status criterion.

8. The system of claim 7, wherein the set of user inputs further comprises processing parameters, wherein executing the signal pipeline further comprises processing the set of neural signals to detect a set of neural spikes according to the processing parameters, wherein the status criterion comprises a neural spike detection criterion.

9. The system of claim 7, wherein the user interface comprises an LED ring.

10. The system of claim 1, wherein the neural probe further comprises a cell container arranged above the set of electrodes, wherein the set of neurons are cultured within the cell container.

11. A system, comprising:
a neural probe comprising a set of electrodes;
an adapter connected to the neural probe, wherein the adapter comprises: a set of inputs connected to the set of electrodes of the neural probe; a set of recording channels; and a set of programmable switches connecting the set of inputs to the set of recording channels;
an interface device connected to the adapter, the interface device configured to:
receive a set of user inputs comprising neural probe specifications; and
execute a signal pipeline, wherein executing the signal pipeline comprises:
controlling the set of programmable switches based on the neural probe specifications to connect two electrodes of the set of electrodes to each recording channel of the set of recording channels; and
controlling the adapter to: record a set of neural signals from the set of recording channels and transmit the set of neural signals to the interface device.

12. The system of claim 11, wherein the neural probe specifications comprise an electrode arrangement, the electrode arrangement comprising a recording electrode identifier and a reference electrode identifier assigned to each recording channel of the set of recording channels.

13. The system of claim 12, wherein the reference electrode identifier is different for each recording channel of the set of recording channels.

14. The system of claim 13, wherein a first recording electrode identifier and a first reference electrode identifier are assigned to a first recording channel in the set of recording channels, wherein, when a sign of a first neural signal recorded from the first recording channel is negative, the first neural signal corresponds to the reference electrode identifier.

15. The system of claim 11, further comprising a remote system configured to receive the set of user inputs, wherein the interface device is communicatively connected to the remote system, wherein the set of user inputs further comprises a user-defined algorithm, wherein executing the signal pipeline further comprises processing the set of neural signals according to the user-defined algorithm.

16. The system of claim 11, wherein the set of neurons comprise optogenetically modified neurons, the neural probe further comprising a set of LEDs configured to transmit light signals to the set of neurons, wherein an LED in the set of LEDs transmits a light signal to a neuron of interest in the set of neurons, and wherein an electrode in the set of electrodes receives an electrical signal from the neuron of interest, wherein the set of user inputs further comprises stimulation parameters, wherein executing the signal pipeline further comprises: controlling the neural probe to drive the set of LEDs based on the set of neural signals and the stimulation parameters.

17. The system of claim 11, further comprising a remote system configured to receive the set of user inputs, wherein the interface device is communicatively connected to the remote system, wherein the set of user inputs further comprises processing parameters, wherein executing the signal pipeline further comprises processing the set of neural signals to detect a set of neural spikes according to the processing parameters.

18. The system of claim 11, further comprising a supplementary device connected to the adapter, wherein the adapter receives a supplementary signal from the supplementary device synchronously with the neural signal received from the neural probe.

19. The system of claim 11, further comprising a stimulation device connected to the adapter, wherein the adapter outputs a supplementary signal to the stimulation device.

20. The system of claim 11, wherein the neural probe is implanted within a brain or implanted on the surface of a brain.
